(19) United States
(12) Patent Application Publication
(10) Pub. No.: US 2022/0248668 A1
(43) Pub. Date: Aug. 11, 2022

(54) ANTIMICROBIAL NANOCLAYS COMPRISING CATIONIC ANTIMICROBIALS, METHOD OF PREPARATION AND USES THEREOF

(71) Applicant: Polyamyna Nanotech Inc., St. John's (CA)

(72) Inventors: Ramesh Kumar Mani, St. John's (CA); Bala Kishan Gorityala, St. John's (CA)

(73) Assignee: Polyamyna Nanotech Inc., St. John's, NL (CA)

(21) Appl. No.: 17/611,234

(22) PCT Filed: May 15, 2020
(86) PCT No.: PCT/IB2020/054651

§ 371 Date: Nov. 15, 2021

Related U.S. Application Data
(60) Provisional application No. 62/848,172, filed on May 15, 2019.

(51) Int. Cl. A01N 25/08; A01N 33/12; A01N 59/20; A01N 59/16; A01N 63/50; A01N 37/22; A01N 37/44; A01P 1/00

(52) U.S. Cl. CPC A01N 25/08; A01N 33/12; A01N 59/20; A01P 1/00; A01N 63/50; A01N 37/22; A01N 37/44; A01N 59/16

(57) ABSTRACT

Described herein are antimicrobial nanoclay comprising two or more cationic antimicrobials. The nanoclay is a phyllosilicate clay and the two or more cationic antimicrobials are individually selected from the group consisting of a quaternary ammonium compound, a metal ion-containing, a cationic chelating agent, a cationic amino acid-based surfactant, a cationic antibiotic, and a cationic compound having a guanidine moiety. The two or more different antimicrobials are incorporated into the nanoclay by a cationic exchange process. Also described are methods for preparing such antimicrobial nanoclays. The antimicrobial nanoclays may be used for conferring antimicrobial activity to an article of manufacture and/or for providing antimicrobial activity to a surface.

### PRIOR RELATED APPLICATION

The instant application claims the benefit of U.S. Provisional Patent Application 62/848,172, filed May 15, 2019, which is incorporated herein by reference in its entirety.

### FIELD OF THE INVENTION

The invention relates to the field of antimicrobial coatings, and more particularly to antimicrobial nanoclays.

### BACKGROUND OF THE INVENTION

With global antimicrobial resistance on rise at an alarming rate, there has been an increase in interest in the role of cleaning for managing the hospital. Numerous studies have demonstrated that surfaces in the rooms of patients are often contaminated with important healthcare-associated pathogens, which causes healthcare-associated infections (HAI) also known as “nosocomial infections. For instance, pathogens such as vancomycin-resistant enterococci (VRE), methicillin-resistant Staphylococcus aureus (MRSA), multidrug-resistant (MDR) Gram-negative bacilli, norovirus, and Clostridium difficile persist in the healthcare environment for days, and it is now recognized that the hospital environment may facilitate the transmission of these dangerous health-care associated pathogens. These pathogens are frequently shed by patients and staff, whereupon they contaminate surfaces for days and increase the risk of acquisition for other patients. Accordingly, it is now clear that contaminated surfaces in healthcare settings cause higher infection risk to the patients.

Antimicrobial coatings and self-disinfecting surfaces are getting wide interest due to the need to develop effective strategies to control infections caused by microbes. For instance, self-disinfecting surfaces can be created by impregnating or coating surfaces with heavy metals (e.g., silver or copper), germicides (e.g., triclosan), or miscellaneous methods (e.g., light-activated antimicrobials). However, since the frequency of antibiotic resistance keeps on increasing, more effective antimicrobial strategies are required, particularly coatings and self-disinfecting surfaces comprising more than one single antimicrobial substance, and preferably antimicrobials from different classes of compounds.

Over the past few years, due to their wide availability, relatively low cost and low environmental impact, nanoclays have been extensively investigated and developed for an array of applications, including as a component for the development of functional nanocomposites having antibacterial activity. For instance, Dai, X., et al. disclose polymer/clay composites with antibacterial properties (Dai, X., et al., Functional Silver Nanoparticle as a Benign Antimicrobial Agent That Eradicates Antibiotic-Resistant Bacteria and Promotes Wound Healing. ACS Appl. Mater. Interfaces 8, 25798-25807 (2016)). Pierchala, M. K. et al. describe the synthesis of an antibacterial, multilayered polylactic acid/halloysite nanoclay composite membrane encapsulated with gentamicin (Pierchala, M. K. et al., Nanotubes in nanofibers: Antibacterial multilayered polylactic acid/halloysite/gentamicin membranes for bone regeneration application. Appl. Clay Sci. 160, 95-105 (2018)). Luo, Y. et al. disclose a synthesis of a smart hydrogel composite with antibacterial activity by using polyvinyl alcohol/chitosan/honey/clay (Luo, Y. et al., Synthesis and Biological Evaluation of Well-Defined Poly(propylene fumarate) Oligomers and Their Use in 3D Printed Scaffolds. Biomacromolecules 17, 690-697 (2016)). Makaremi, M. et al. have employed two different types of halloysite nanoclays for preparing antibacterial pectin/nanoclay composite showing activity against different strains of Gram-positive and Gram-negative bacteria (Makaremi, M. et al. Effect of Morphology and Size of Halloysite Nanotubes on Functional Pectin Bionanocomposites for Food Packaging Applications. ACS Appl. Mater. Interfaces 9, 17476-17488 (2017)).

Additional antimicrobials phyllosilicate compositions are also described in the following patent documents: WO2010141070, U.S. Pat. No. 5,876,738, EP1896529, JP1990019308, JP 2009067693, KR1020130053175, KR101303530 and TW200826978.

Others have also manufactured clay-based coatings with potential antimicrobial properties. For instance Armstrong et al. have formulated epoxy-polyester powder coatings containing silver-modified nanoclays (Armstrong, G. et al. Formulation of epoxy-polyester powder coatings containing silver-modified nanoclays and evaluation of their antimicrobial properties. Polym. Bull. 68, 1951-1963 (2012)), Giraldo Mejia et al. have developed silver-rich nanocomposite thin coating, loaded with organically modified clay nanoparticles, and tested its structure, morphology, thermal and antimicrobial properties (Giraldo Mejia, H. F. et al. Epoxy-silica/clay nanocomposite for silver-based antibacterial thin coatings: Synthesis and structural characterization. J. Colloid Interface Sci. 508, 332-341 (2017)), and Serrano et al. have incorporated silver (Ag) to organo-modified layered silicate additives and these were tested against S. aureus, MRSA, VRE, K. pneumoniae, P. aeruginosa, A. baumannii and E. coli (Monte-Serrano, M et al. Effective Antimicrobial Coatings Containing Silver-Based Nanoclays and Zinc Pyrithione. J Microb Biochem Technol 7, 398-403 (2015).

However, existing antimicrobial clay-based compositions or coatings are limited by the fact they do not combine multiple antimicrobials. To the best knowledge of the Applicant, a combinational approach in a nanosystem has never been achieved or reported before. Accordingly, it would be desirable to combine at least two different antimicrobials in the same nanoclay in order to increase efficacy against multiple types of microbes, to increase efficacy against resistant microorganisms and, hopefully, obtain a synergistic effect where combined activity two agents (or more) is greater than the sum of their individual activities.

Accordingly, there is a need for new and improved biocidal materials exhibiting the following properties: facile synthesis, long-term stability, water insolubility, nontoxicity and broad-spectrum biocidal activity over short contact times.

There is more particularly a need for antimicrobial nanoclays comprising two or more antimicrobials. There is also a need for antimicrobial nanoclays comprising two or more antimicrobials from at least two different groups or two different classes of compounds.

There is also a need for nanoclays with antimicrobial efficacy against antibiotic-resistant bacteria.

There is also a need for antimicrobial nanoclays effective against a wide range of microbes such as bacteria, viruses, algae, yeasts and mold.

There is also a need for nanoclay-based coatings with antimicrobial properties for conferring antimicrobial activity to a surface.

There is also a need for innovative synthesis pathways that would result in novel nanoclay based materials, which could be used in the manufacture of polymer-nanoclay composites with enhanced physio-chemical and biological properties.

The present invention addresses these needs and other needs as it will be apparent from review of the disclosure and description of the features of the invention hereinafter.

### BRIEF SUMMARY OF THE INVENTION

According to one aspect, the invention relates to antimicrobial nanoclay comprising: (i) a phyllosilicate clay; and (ii) two or more cationic antimicrobials, wherein said two or more cationic antimicrobials are individually selected from the group consisting of a quaternary ammonium compound, a metal ion, a cationic chelating agent, a cationic amino acid-based surfactant, a cationic antibiotic, and a cationic compound having a guanidine moiety.

According to another aspect, the invention relates to the use of an antimicrobial nanoclay as defined herein for conferring antimicrobial activity to an article of manufacture and/or for providing antimicrobial activity to a surface. Related aspects concern an article of manufacture comprising an antimicrobial nanoclay as defined herein and a method for conferring antimicrobial activity to a surface, comprising contacting the surface with an antimicrobial nanoclay as defined herein.

Another aspect of the invention relates to a method for preparing an antimicrobial nanoclay as defined herein.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE DRAWINGS

FIG. 1: Schematic representation of mono cationic exchange process.

FIG. 2: Schematic representation of dual cationic exchange process.

FIG. 3: Schematic representation of triple cationic exchange process.

FIG. 4: TGA weight loss thermograms of (a) BN-BDMHAC, (b) BN-Ag-BDMHAC and (c) BN-Ag. TGA derivative weight loss plots of (d) BN-BDMHAC, (e) BN-Ag-BDMHAC and (f) BN-Ag.

FIG. 5: XRD plots of (a) bentonite, (b) BN-Ag, (c) BN-BDMHAC and (d) BN-Ag-BDMHAC.

FIG. 6: FTIR spectra of (a) bentonite, (b) BN-BDMHAC, and (d) BDMHAC.

FIG. 7: EDX plot of BN-Ag-BDMHAC sample.

FIG. 8: XRD plots of (a) bentonite, (b) BN-NM, (c) BN-CHL and (d) BN-CHL-NM (material 29).

FIG. 9: TGA derivative weight loss peaks of plots of chlorohexidine, Neomycin sulfate and BN-CHL-NM (material 29).

FIG. 10: FTIR spectra of (a) bentonite, (b) chlorohexidine, (c) neomycin sulfate and (d) BN-CHL-NM (material 29).

FIG. 11: TGA weight loss thermograms of (a) BN-Cu, (b) BN-Ag—Cu and (c) BN-Ag. TGA derivative weight loss plots of (d) BN-Cu, (e) BN-Ag—Cu and (f) BN-Ag.

FIG. 12: EDX spectra of material 18 (BN-Ag-Ga).

FIG. 13: EDX spectrum of BN-Ag-Cu-Ga sample (material 36).

FIG. 14: FTIR spectrum of BN-Ag-Cu-Ga sample (material 36).

FIG. 15: TGA weight loss and derivative weight loss thermograms of BN-Ag, BN-Cu, BN-Ga and BN-Ag-Cu-Ga samples (material 36).

FIG. 16: XRD spectra of (a) montmorillonite (MMT) and (b) MMT+NM-CHL (material 31).

FIG. 17: Derivative weight loss thermogram of (a) MMT, (b) chlorohexidine, (c) neomycin sulfate and (d) MMT-NM-CHL (material 31).

FIG. 18: FTIR spectrum of MMT-NM-CHL (material 31).

FIG. 19: XRD spectra of (a) halloysite (MMT) and (b) HS-NM-CHL (material 32).

FIG. 20: Derivative weight loss thermogram of (a) halloysite, (b) chlorohexidine, (c) neomycin sulfate and (d) HS-NM-CHL (material 32).

FIG. 21: FTIR spectrum of HS-NM-CHL (material 32).

FIG. 22: XRD spectra of (a) surface modified clay (SMC) and (b) SMC-NM-CHL.

FIG. 23: Derivative weight loss thermogram of (a) SMC, (b) chlorohexidine, (c) neomycin sulfate and (d) SMC-NM-CHL (30).

FIG. 24: FTIR spectrum of SMC-NM-CHL (material 30).

### DETAILED DESCRIPTION OF EMBODIMENTS

In the following description of the embodiments, references to the accompanying drawings are illustrations of an example by which the invention may be practiced. It will be understood that other embodiments may be made without departing from the scope of the invention disclosed. Unless defined otherwise, all technical and scientific terms used herein have the same meaning as commonly understood by one of ordinary skill in the art to which the invention belongs.

As described herein, the present inventors have employed a combinational approach in order to obtain a powerful antimicrobial additive material. Particularly, combinational approach was used so as to achieve a broadened antibacterial spectrum via a “synergy” effect, i.e. to obtain a combined effect of two agents greater than the sum of the individual activities of the two agents.

### Antimicrobial Nanoclays

One particular aspect of the present invention relates to an antimicrobial nanoclay comprising a phyllosilicate clay and at least two cationic antimicrobials.

As used herein, the term “nanoclay” refers to clay mineral(s) that have been enhanced or optimized for a particular use or application. In accordance with the present invention, the nanoclay comprises antimicrobial properties. Preferably, the nanoclay comprises layered silicates such that, when a suitable clay is added in a matrix, these layers are able to separate as nanosheets and form either intercalated or exfoliated nanocomposites. The term “nanoclay” as used herein encompasses clays before and/or after exfoliation.

According to the present invention, the nanoclay is a phyllosilicate clay. As used herein, the term “phyllosilicate clay”, also known as “sheet silicate” refers to minerals that includes the micas, chlorite, serpentine, talc, and the clay minerals. In embodiments, the invention encompasses clays of montmorillonite, kaolinite, bentonite, smectite, hectorite, sepiolite, gibbsite, dickite, nacrite, saponite, halloysite, vermiculite, mica type, chlorite, illite, kalonite-serpentine group, nontronite, attapulgite mixtures thereof, as well as nanoclays obtained therefrom.

The invention encompasses also nanoclays comprising organic and/or inorganic surface modification(s), including but not limited to silver, copper, gallium, benzalkonium chloride, etc. Particular examples of modified nanoclays include, but are not limited to, Nanomer® MMT Clay (Sigma Aldrich, which contains 0.5-5 wt. % aminopropyltriethoxysilane, 15-35 wt % octadecylamine), and Montmorillonite-surface modified (contains 25-30 wt. % trimethyl stearyl ammonium), Montmorillonite-surface modified (contains 35-45 wt. % dimethyl dialkyl (C14-C18) amine) and Closide30B™ (alkyl quaternary ammonium salt bentonite).

In some embodiments, the nanoclay is selected from the group consisting of bentonite, surface modified clays (e.g. Nanomer®Clay), montmorillonite and halloysite.

The present invention also encompasses using different types of clays (e.g. mixture of two or more different clays).

As used herein, the term “antimicrobial” or “antimicrobial properties” or “antimicrobial activity” refers to killing or inhibiting growth of microbes including, but not limited to, bacteria, viruses, algae, yeasts and mold. In preferred embodiments, the nanoclay possesses antimicrobial activity of at least 60%, or at least 70%, or at least 80%, or at least 90%, preferably at least 99%, and more preferably of at least 100%, as measured by any suitable antimicrobial efficacy testing.

In selected embodiments, the least two cationic antimicrobial comprises at least one antibacterial agent. As used herein, the term “antibacterial agent” refers to any compound having antibacterial activity including, but not limited metal ions, quaternary ammonium compounds, cationic chelating agents, cationic antibiotics, cationic amino acid-based surfactants, compounds having a cationic guanidine moiety, and cationic antimicrobial peptides.

In particular embodiments, the microbe or bacteria is a Gram-positive bacteria. In other embodiments, the microbe is a Gram-negative bacteria. Examples of Gram-positive bacteria include, but are not limited to, many well-known genera such as Staphylococcus, Streptococcus, Enterococcus and Bacillus. Examples of Gram-negative bacteria include, but are not limited to, Escherichia coli, Salmonella, Shigella, and other Enterobacteriaceae, Pseudomonas, Moraxella, Helicobacter, Stenotrophomonas, Bdellovibrio, acetic acid bacteria, Legionella and alpha-proteobacteria as Wolbachia and numerous others. Other notable groups of Gram-negative bacteria include the cyanobacteria, spirochaetes, green sulfur and green non-sulfur bacteria. Examples of yeasts include, but are not limited to, Saccharomyces cerevisiae and pathogenic yeast such as Candida. In preferred embodiments, the anodized metal products according to the invention have an antimicrobial activity against one or more of the following pathogens: Staphylococcus aureus, Bacillus subtilis, Escherichia coli, Pseudomonas aeruginosa, and Candida albicans.

In selected embodiments, the metal ion-containing compound having antimicrobial/antibacterial activity is a compound comprising a metal ion selected from silver (Ag), titanium (Ti), cobalt (Co), nickel (Ni), zinc (Zn), molybdenum (Mo), gallium (Ga), copper (Cu), zirconium (Zr), tin (Sn), lead (Pb), and iron (Fe).

In selected embodiments, the quaternary ammonium compound having antimicrobial/antibacterial activity is selected from the group consisting of benzalkonium compounds having alkyl chains C8 to C18; quaternary ammonium compounds having an aromatic ring with nitrogen, chlorine and alkyl groups; quaternary ammonium compounds having a dialkylmethyl amino with twin chains; and polymeric quaternary ammonium compounds. Particular examples encompassed quaternary ammonium compounds include, but are not limited to, Alkyl Dimethyl Benzyl Ammonium Chloride (ADBAC), Didecyl Dimethyl Ammonium Chloride (DDAC), Benzyldimethyl(2-dodecyloxyethyl)-ammonium chloride, Benzyldimethyl(2-hydroxyethyl)ammonium chloride, benzyldimethyl (hexadecylcarbamoylmethyl)ammonium chloride, benzyldimethyl (tetradecylcarboamoylmethyl)ammonium chloride, benzyloxycarbonylmethyl-trimethyl-ammonium chloride, bis-(2-hydroxyethyl)-ciannamyl(2-dodecyloxymethyl)ammonium chloride, Benzyltriethylammonium chloride, Tetramethylammonium chloride, Tetramethylammonium iodide, Tetraethylammonium hydroxide, Tetramethylammonium hydroxide, Benzyltrimethylammonium hydroxide, Dimethyldioctadecylammonium chloride, Dodecyltrimethylammonium choride, Trimethylphenylammonium chloride, Octadecyltrimethyl ammonium bromide, Tetrabutyl ammonium bromide, Tetramethylammonium nitrate, Tetrabutylammonium hydroxide, Didodecyldimethyl ammonium bromide, Didodecyldimethylammonium chloride, Dimethyldioctadecyl ammonium bromide, (2-(Methacryloyloxy)ethyl)trimethylammonium chloride, Dioctyl dimethyl ammonium chloride, Tetrapropylammonium chloride, Didecyldimethylammonium chloride, Bezyldodecyldimethyl ammonium bromide, Diallyl dimethyl ammonium chloride, Benzalkonium bromide, Ammonium bromide, Benzyltributylammonium chloride, Octyldecyl dimethyl ammonium chloride, Tetrabutylammonium hydrogen sulfate, Tetrabutylammonium tribromide, Methyltributylammonium chloride, Bis(hydrogenated tallow)dimethylammonium chloride, N-Alkyl dimethyl benzyl ammonium chloride, Tetrabutylammonium fluoride trihydrate.

As used herein, the term “cationic chelating agent” refers to a chelating agent comprising at least one cationic moiety including, but not limited to, amine groups. In selected embodiments, the cationic chelating agent having antimicrobial/antibacterial activity is selected from the group consisting of ethyleneglycoltetraacetate (EGTA), ethylenediaminetetraacetate (EDTA), deferrioxamine B (DFO, desferal), D-penicillamine (DPA), 1,10-phenanthroline (phen), and zinc pyrithione. Additional chelating agents that might be considered are 2,3-dimeracapto-1-propanol (BAL) and 2,3-dimercapto-1-propanesulfonic acid (DMPS).

In selected embodiments, the cationic antibiotic having antimicrobial/antibacterial activity is selected from the classes consisting of aminoglycosides, ansamycins, carbacephems, carbapenems, cephalosporins (first, second, third, fourth or fifth generation), glycopeptides, lincosamides, lipopeptides, macrolides, monobactams, nitrofurans, oxazolidinones, penicillins, polypeptides, quinolines, fluoroquinolines, sulfonamidea, and tetracyclines.

Particular examples of envisioned antibiotics include, but are not limited to, clofazimine, dapsone, capreomycin, cycloserine, ethambutol, ethionamide, isoniazid, pyrazinamide, rifampicin (rifampin), rifabutin, rifapentine, streptomycin, arsphenamine, chloramphenicol, Fosfomycin, fusidic acid, niclosamide, metronidazole, mupirocin, platensimycin, quinupristin, dalfopristin, thiamphenicol, tigecycline, tinidazole, and trimethoprim.

In selected embodiments, the cationic compound having a guanidine moiety. Particular envisioned examples include, but are not limited to, chlorhexidine gluconate (CHG), polyhexamethylene guanidine hydrochloride, guanidine hydrochloride, proguanil, cycloguanil, chlorproguanil, metformin, famotidine, nemaucing, rhodostreptomycin A, rhodostreptomycin B, synoxaazolidinone A, synoxaazolidinone B and chemical derivatives thereof.

In selected embodiments, the least two cationic antimicrobial comprises at least one antifungal agent. As used herein, the term “antifungal agent” refers to any compound having antifungal activity including, but not limited to polyene (Natamycin, rimocidin, candicin, etc.), imidazole, triazole, thiazole (miconazole, fluconazole, abafungin, etc.), allylamines (terbinafine, naftifine, etc.), echinocandin (caspofungin, micafungin, etc.), and other antifungals such as clotrimazole, fluconazole, ravuconazole, voriconazole, itraconazole, Posaconazole, 5-flucytosine, ciclopirox, griseofulvin, etc.

In selected embodiments, the least two cationic antimicrobial comprises at least one antiviral. As used herein, the term “antiviral” refers to any compound having the potential of destruction of a virus or the potential to inhibit the penetration of the virus into a host cell. Examples include, but are not limited to, ritonavir, ribavirin, nelfinavir, acyclovir, penciclovir, cidofovir, adenine arabinoside, methisazone, idoxuridine, niclosamide, sofosbuvir, emricasan, mefloquine, palonosetron, inosine, lamivudine, stavudine, zidovudine, abacavir, didanosine, tenofovir, emtricitabine, efavirenz, nevirapine, indinavir, saquinavir amantadine and combination thereof

It is also envisioned according to the present invention to modify a “non-cationic” molecule to make it “cationic” while still retaining the antimicrobial activity so it ca be used according to the principles of present invention. For instance, it may be possible to introduce a cationic moiety into a molecule (e.g. synthesizing a prodrug antibiotic) such that the cationic-modified antimicrobial molecule can be incorporated into the nanoclay using mono, dual or triple combinations, either independently or in combination with claimed cationic antimicrobials, to provide additive or synergistic effect.

According to the present invention, the combination of two or more antimicrobials in a nanoclay system exerts superior antimicrobial efficacy. While not wishing to be bound to a particular theory or hypothesis, the inventors believe that microbial susceptibility to multiple anti-microbial nanoclay systems is through a combination of mechanisms, with many of those related to the cell membrane and other bacterial cell components. As will be appreciated by those of skill in the art, multiple modes of action are preferred in order to counter multi-drug resistance bacteria. For example, the mode of action of quaternary ammonium compounds (QACs), including benzalkonium chloride, involves the perturbation and disruption of the membrane bilayers by the alkyl chains and disruption of charge distribution of the membrane by the charged nitrogen. QACs are membrane-active agents that interact with the cytoplasmic membrane of bacteria and the plasma membrane of yeast. Their hydrophobic activity also makes them effective against lipid-containing viruses. QACs also interact with intracellular targets and bind to DNA. They can also be effective against non-lipid-containing viruses and spores, depending on the product formulation. At low concentrations (0.5 to 5 mg/liter), they are algistatic, bacteriostatic, tuberculostatic, sporostatic, and fungistatic. In contrast, electrically generated silver ion exert their antibacterial effect by inducing bacteria into “active but nonculturable” (ABNC) state, in which the mechanisms required for the uptake and utilization of substrates leading to cell division are disrupted at the initial stage, which causes the bacterial cells to undergo morphological changes and eventually die.

For instance, as demonstrated in the Exemplification section, combination of quaternary ammonium compounds and silver salts result in superior antimicrobial efficacies against a range of multi-drug resistant pathogens. Similarly, combinations of quaternary ammonium and copper, and quaternary ammonium and EDTA also provided nanocomposites with significant antimicrobial activities.

Accordingly, the invention provides for the co-localization of two or more, for example, two, three, four or more different anti-microbial agents which can exert multiple different effects on microbial cells essentially simultaneously.

According to one particular embodiment, the antimicrobial nanoclay comprises two or more cationic antimicrobials from one or more different groups or classes of compounds (e.g. two metal ions, one metal ion and one quaternary ammonium compound, or one antibiotic and one antiseptic, etc.). According to one particular embodiment, these at least three different cationic antimicrobials are independently selected from the group consisting of quaternary ammonium compound, a metal ion, a chelating agent, a cationic amino acid-based surfactant, a cationic antibiotic, and a cationic compound having a guanidine moiety.

According to particular embodiments, the antimicrobial nanoclay comprises three or more cationic antimicrobials from one or more different groups or classes of compounds. According to one particular embodiment, these at least three different cationic antimicrobials are independently selected from the group consisting of quaternary ammonium compound, a metal ion, a chelating agent, a cationic amino acid-based surfactant, a cationic antibiotic, and a cationic compound having a guanidine moiety.

It is within the skill persons to obtain an antimicrobial nanoclay in accordance with the present invention comprising more than two or three cationic antimicrobials, from the same or different group or classes. Accordingly, the present invention encompasses the use of two or three, or four or five or six or more cationic antimicrobials, from the same or different group or classes.

In some embodiments of the invention, the antimicrobial nanoclay comprises: (i) a first antimicrobial agent selected from the group consisting of a quaternary ammonium compound, a metal ion, a cationic chelating agent, a cationic amino acid-based surfactant, a cationic antibiotic, and a cationic compound having a guanidine moiety; and (ii) a second antimicrobial agent, different from the first antimicrobial agent, selected from the group consisting of: a quaternary ammonium compound, a metal ion, a cationic chelating agent, a cationic amino acid-based surfactant, a cationic antibiotic, and a cationic compound having a guanidine moiety.

In some embodiments of the invention, the antimicrobial nanoclay further comprises a third antimicrobial agent, different from the first antimicrobial agent and the second antimicrobial agent, selected from the group consisting of a quaternary ammonium compound, a metal ion, a cationic chelating agent, a cationic amino acid-based surfactant, a cationic antibiotic, and a cationic compound having a guanidine moiety.

In some other embodiments of the invention, the antimicrobial nanoclay further comprises a fourth antimicrobial agent, different from the first, second and third antimicrobial agents, selected from the group consisting of a quaternary ammonium compound, a metal ion, a cationic chelating agent, a cationic amino acid-based surfactant, a cationic antibiotic, and a cationic compound having a guanidine moiety.

According to the present invention, the first antimicrobial agent does not necessarily need to be of a different class of antimicrobial from the second antimicrobial agent. That is, the first antimicrobial agent and the second antimicrobial agent may be of the same or from two different classes. For instance, the first antimicrobial agent and the second antimicrobial agent may be two different quaternary ammonium compounds.

In some embodiments of the invention, the first antimicrobial agent and the second antimicrobial agent different and are independently silver, copper, gallium, zinc pyritione, benzalkonium chloride, polymyxin E, niclosamide, ethylenediaminetetraacetic acid, benzyldimethyltetradecylammonium chloride, neomycin, or chlorohexidine.

In particular embodiments, the antimicrobial nanoclay is any one from Material Number 17 to 144 listed below:

| Material Number | R | R₁ | R₂ | R₃ |
|---|---|---|---|---|
| 17 | BN | Ag | Cu | — |
| 18 | BN | Ag | Ga | — |
| 19 | BN | Cu | Ga | — |
| 20 | BN | Cu | ZnPy | — |
| 21 | BN | Ag | ZnPy | — |
| 22 | BN | Ag | BAC | — |
| 23 | BN | Cu | BAC | — |
| 24 | BN | Ag | BDMHAC | — |
| 25 | BN | Ag | BDMTAC | — |
| 26 | BN | Cu | BDMHAC | — |
| 27 | BN | Cu | BDMTAC | — |
| 28 | BN | BAC | BDMHAC | — |
| 29 | BN | NM | CHL | — |
| 30 | SMC | NM | CHL | — |
| 31 | MMT | NM | CHL | — |
| 32 | HS | NM | CHL | — |
| 33 | BN | PMX | NCL | — |
| 34 | BN | Ag | EDTA | — |
| 35 | BN | BAC | EDTA | — |
| 36 | BN | Ag | Cu | Ga |
| 37 | BN | Ag | BAC | ZnPy |
| 38 | BN | Ag | BDMHAC | ZnPy |
| 39 | BN | BAC | BDMHAC | BDMTAC |
| 40 | BN | NM | CHL | EDTA |
| 46 | MMT | Ag | Cu | — |
| 47 | MMT | Ag | Ga | — |
| 48 | MMT | Cu | Ga | — |
| 49 | MMT | Cu | ZnPy | — |
| 50 | MMT | Ag | ZnPy | — |
| 51 | MMT | Ag | BAC | — |
| 52 | MMT | Cu | BAC | — |
| 53 | MMT | Ag | BDMHAC | — |
| 54 | MMT | Ag | BDMTAC | — |
| 55 | MMT | Cu | BDMHAC | — |
| 56 | MMT | Cu | BDMTAC | — |
| 57 | MMT | BAC | BDMHAC | — |
| 58 | MMT | Ag | EDTA | — |
| 59 | MMT | BAC | EDTA | — |
| 60 | MMT | PMX | NCL | — |
| 61 | MMT | Ag | Cu | Ga |
| 62 | MMT | Ag | BAC | ZnPy |
| 63 | MMT | Ag | BDMHAC | ZnPy |
| 64 | MMT | BAC | BDMHAC | BDMTAC |
| 65 | MMT | NM | CHL | EDTA |
| 66 | SMC | Ag | Cu | — |
| 67 | SMC | Ag | Ga | — |
| 68 | SMC | Cu | Ga | — |
| 69 | SMC | Cu | ZnPy | — |
| 70 | SMC | Ag | ZnPy | — |
| 71 | SMC | Ag | BAC | — |
| 72 | SMC | Cu | BAC | — |
| 73 | SMC | Ag | BDMHAC | — |
| 74 | SMC | Ag | BDMTAC | — |
| 74 | SMC | Cu | BDMHAC | — |
| 75 | SMC | Cu | BDMTAC | — |
| 76 | SMC | BAC | BDMHAC | — |
| 77 | SMC | Ag | EDTA | — |
| 78 | SMC | BAC | EDTA | — |
| 79 | SMC | PMX | NCL | — |
| 80 | SMC | Ag | Cu | Ga |
| 81 | SMC | Ag | BAC | ZnPy |
| 82 | SMC | Ag | BDMHAC | ZnPy |
| 83 | SMC | BAC | BDMHAC | BDMTAC |
| 84 | SMC | NM | CHL | EDTA |
| 85 | HS | Ag | Cu | — |
| 86 | HS | Ag | Ga | — |
| 87 | HS | Cu | Ga | — |
| 88 | HS | Cu | ZnPy | — |
| 89 | HS | Ag | ZnPy | — |
| 90 | HS | Ag | BAC | — |
| 91 | HS | Cu | BAC | — |
| 92 | HS | Ag | BDMHAC | — |
| 93 | HS | Ag | BDMTAC | — |
| 93 | HS | Cu | BDMHAC | — |
| 94 | HS | Cu | BDMTAC | — |
| 95 | HS | BAC | BDMHAC | — |
| 96 | HS | Ag | EDTA | — |
| 97 | HS | BAC | EDTA | — |
| 98 | HS | PMX | NCL | — |
| 99 | HS | Ag | Cu | Ga |
| 100 | HS | Ag | BAC | ZnPy |
| 101 | HS | Ag | BDMHAC | ZnPy |
| 102 | HS | BAC | BDMHAC | BDMTAC |
| 103 | HS | NM | CHL | EDTA |
| 104 | KL | Ag | Cu | — |
| 105 | KL | Ag | Ga | — |
| 106 | KL | Cu | Ga | — |
| 107 | KL | Cu | ZnPy | — |
| 108 | KL | Ag | ZnPy | — |
| 109 | KL | Ag | BAC | — |
| 110 | KL | Cu | BAC | — |
| 111 | KL | Ag | BDMHAC | — |
| 112 | KL | Ag | BDMTAC | — |
| 113 | KL | Cu | BDMHAC | — |
| 114 | KL | Cu | BDMTAC | — |
| 115 | KL | BAC | BDMHAC | — |
| 116 | KL | Ag | EDTA | — |
| 117 | KL | BAC | EDTA | — |
| 118 | KL | PMX | NCL | — |
| 119 | KL | NM | CHL | — |
| 120 | KL | Ag | Cu | Ga |
| 121 | KL | Ag | BAC | ZnPy |
| 122 | KL | Ag | BDMHAC | ZnPy |
| 123 | KL | BAC | BDMHAC | BDMTAC |
| 124 | KL | NM | CHL | EDTA |
| 125 | LP | Ag | Cu | — |
| 126 | LP | Ag | Ga | — |
| 127 | LP | Cu | Ga | — |
| 128 | LP | Cu | ZnPy | — |
| 129 | LP | Ag | ZnPy | — |
| 130 | LP | Ag | BAC | — |
| 131 | LP | Cu | BAC | — |
| 132 | LP | Ag | BDMHAC | — |
| 133 | LP | Ag | BDMTAC | — |
| 134 | LP | Cu | BDMHAC | — |
| 135 | LP | Cu | BDMTAC | — |
| 136 | LP | BAC | BDMHAC | — |
| 137 | LP | Ag | EDTA | — |
| 138 | LP | BAC | EDTA | — |
| 139 | LP | PMX | NCL | — |
| 140 | LP | NM | CHL | — |
| 141 | LP | Ag | BAC | ZnPy |
| 142 | LP | Ag | BDMHAC | ZnPy |
| 143 | LP | BAC | BDMHAC | BDMTAC |
| 144 | LP | NM | CHL | EDTA |

wherein R represents the phyllosilicate nanoclay, R₁ represents a first antimicrobial, R₂ represents a second antimicrobial, R₃ represents a third antimicrobial; and

wherein BN=Bentonite; SMC=Surface modified clay; MMT=Montmorillonite; HS=Halloysite; KL=Kaolinite; LP=Laponite; Ag=Silver; Cu=Copper; Ga=Gallium; ZnPy=Zinc pyrithione; BAC=Benzalkonium chloride; BDMHAC=Benzyldimethylhexadecylammonium chloride; BDMTAC Benzyldimethyltetradecylammonium chloride; NM=Neomycin; CHL=Chlorohexidine; PMX=Polymyxin E; NCL=Niclosamide; EDTA=Ethylenediaminetetraacetic acid.

In one particular embodiment, the phyllosilicate clay is bentonite, the first antimicrobial agent is silver and the second antimicrobial agent is copper.

In another particular embodiment, the phyllosilicate clay is bentonite, the first antimicrobial agent is silver and the second antimicrobial agent is gallium.

In another particular embodiment, the phyllosilicate clay is bentonite, the first antimicrobial agent is copper and the second antimicrobial agent is gallium.

In another particular embodiment, the phyllosilicate clay is bentonite, the first antimicrobial agent is copper and the second antimicrobial agent is zinc pyrithione.

In another particular embodiment, the phyllosilicate clay is bentonite, the first antimicrobial agent is silver and the second antimicrobial is zinc pyrithione.

In another particular embodiment, the phyllosilicate clay is bentonite, the first antimicrobial agent is silver and the second antimicrobial agent is Benzalkonium chloride.

In another particular embodiment, the phyllosilicate clay is bentonite, the first antimicrobial agent is copper and the second antimicrobial agent is benzalkonium chloride.

In another particular embodiment, the phyllosilicate clay is bentonite, the first antimicrobial agent is silver and the second antimicrobial agent is benzyldimethylhexadecylammonium chloride.

In another particular embodiment, the phyllosilicate clay is bentonite, the first antimicrobial agent is silver and the second antimicrobial agent is benzyldimethyltetradecylammonium chloride.

In another particular embodiment, the phyllosilicate clay is bentonite, the first antimicrobial agent is copper and the second antimicrobial agent is benzyldimethylhexadecylammonium chloride.

In another particular embodiment, the phyllosilicate clay is bentonite, the first antimicrobial agent is copper and the second antimicrobial agent is benzyldimethyltetradecylammonium chloride.

In another particular embodiment, the phyllosilicate clay is bentonite, the first antimicrobial agent is benzalkonium chloride and the second antimicrobial agent is benzyldimethylhexadecylammonium chloride.

In another particular embodiment, the phyllosilicate clay is bentonite, the first antimicrobial agent is neomycin and the second antimicrobial agent is chlorohexidine.

In another particular embodiment, the phyllosilicate clay is a surface modified clay, the first antimicrobial agent is neomycin and the second antimicrobial agent is chlorohexidine.

In another particular embodiment, the phyllosilicate clay is montmorillonite, the first antimicrobial agent is neomycin and the second antimicrobial agent is chlorohexidine.

In another particular embodiment, the phyllosilicate clay is halloysite, the first antimicrobial is neomycin and the second antimicrobial is chlorohexidine.

In another particular embodiment, the phyllosilicate clay is bentonite, the first antimicrobial is polymyxin E and the second antimicrobial is niclosamide.

In another particular embodiment, the phyllosilicate clay is bentonite, the first antimicrobial is silver and the second antimicrobial is ethylenediaminetetraacetic acid.

In another particular embodiment, the phyllosilicate clay is bentonite, the first antimicrobial agent is benzalkonium chloride and the second antimicrobial agent is ethylenediaminetetraacetic acid.

In another particular embodiment, the phyllosilicate clay is bentonite, the first antimicrobial is silver, the second antimicrobial is copper and the third antimicrobial is gallium.

In another particular embodiment, the phyllosilicate clay is bentonite, the first antimicrobial is silver, the second antimicrobial is benzalkonium chloride and the third antimicrobial is zinc pyrithione.

In another particular embodiment, the phyllosilicate clay is bentonite, the first antimicrobial is silver, the second antimicrobial is benzyldimethylhexadecylammonium chloride and the third antimicrobial is zinc pyrithione.

In another particular embodiment, the phyllosilicate clay is bentonite, the first antimicrobial is benzalkonium chloride, the second antimicrobial is benzyldimethylhexadecylammonium chloride and the third antimicrobial is benzyldimethyltetradecylammonium chloride.

In another particular embodiment, the phyllosilicate clay is bentonite, the first antimicrobial is neomycin, the second antimicrobial is chlorohexidine and the third antimicrobial is ethylenediaminetetraacetic acid.

It is also envisioned according to the present invention to introduce additional biologically active compounds or drugs to the nanoclay including, but not limited to, niclosamide.

It may also be possible to introduce additional non-biologically active compounds or additives to the antimicrobial nanoclay in order to enhance various of its properties. For instance, such cationic additives may be used to improve mechanical properties (e.g. zinc stearate), thermal properties, electrical properties, barrier properties, rheology properties, scavenging properties (e.g. oleic acid), exfoliation properties (e.g. phosphonium, tributyl tetradecyl compounds), fire retardant properties, plasticize properties (e.g. phthalates), permeability properties and color properties. Other examples include stabilizer and surfactants (e.g. tetraphenylphosphonium bromide, n-pentyl-triphenylphosphonium bromide, etc.).

### Methods of Preparation of Antimicrobial Nanoclays

In accordance with the present invention, the antimicrobial nanoclay is preferably obtained by a cationic exchange process. Particularly, the two or more antimicrobials are incorporated into the nanoclay using a cationic exchange process.

Accordingly, in accordance such process, the antimicrobials need to be cationic in order to be exchanged into the nanoclay system. Indeed, most clays, including bentonite and montmorillonite, have metal cations inside their lattice space. Therefore, in accordance with the present invention, it is possible to replace these metal cations other cations. The present invention thus replaces cations in the clay by cationic antimicrobials to obtain a nanoclay with antimicrobial properties.

Various methods, techniques, conditions, clays, antimicrobials, etc. can be used to obtain an antimicrobial nanoclay in accordance with the invention.

As is known, different clays have different lattice space (d-spacing that can be measured using XRD, as exemplified hereinafter). When the d-space is greater, the cation exchange process becomes easier.

As known to those of skill in the art, different nanoclays have different d spacing. For example, the d spacing is 1 nm for bentonite and up to 3 nm for some surface modified clays. The larger the d space, the greater the chance for the cation exchange process to occur as the exchangeable cations can get into the d spacing more easily.

Similarly, smaller cationic antimicrobials will exchange more easily in nanoclay when compared to larger cationic antimicrobials, especially in nanoclays where the d-spacing is small, for example, approximately 1 nm or less. Furthermore, as the nanoclay is inorganic, inorganic cations (such as for example Ag⁺) may exchange more freely into the nanoclay than organic cations (such as for example BAC⁺).

Table 11 hereinafter provides d-spacing of various nanoclays that have successfully been modified with various antimicrobials in accordance with the present invention. As can be appreciated, variation of d-spacing can be used as an indicator that antimicrobials(s) have successfully been incorporated into the clay.

In order to ease the cationic exchange process, it is preferable to lower the pH of the clay by dispersing the clay in an aqueous acidic solution to increase the cation exchange. In embodiment, the pH is between about 3 to about 7, preferably about 4 to about 5, or about 5. In some embodiments, the pH is adjusted using nitric acid, as most of the metal compounds used as antimicrobial agents are in the form of nitrate salts. For example, if we use HCl to adjust the pH, the antimicrobial silver would interact with the Cl— of HCl, and form silver chloride. As such, the type of acid that is used is dependent on the metal salts (or the anion of the antimicrobial).

According to one particular embodiment, the method for preparing an antimicrobial nanoclay, comprises the steps of:
providing an aqueous solvent having a pH of between about 3 to about 7;
dispersing a phyllosilicate clay in the aqueous solvent to obtain a dispersed clay solution; and
mixing two or more cationic antimicrobials into the clay solution to obtain an antimicrobial clay mixture;
   wherein said antimicrobials are selected from the group consisting of a quaternary ammonium compound, a metal ion, a chelating agent, a cationic amino acid-based surfactant, a cationic antibiotic, and a cationic compound having a guanidine moiety.

In embodiments, the clay is dispersed in the aqueous solvent at about 0.1 wt % to about 60 wt % of the solvent (or about 1 wt % to about 40 wt %, or about 2 wt % to about 25 wt %, or about 3 wt % to about 15 wt %). One example of a suitable aqueous solvent is water, preferably water that is free of contaminants and/or salts such as distilled water. Other suitable aqueous solvent may include protic polar solvents such as alcohols at about 0.1% w/w to about 99% w/w, (e.g. ethyl alcohol, methyl alcohol, butanol, propanol, 2-Ethylbutanol, 3-Methyl-3-pentanol, 2,2-Dimethylcyclopropanol, 1,2-Ethanediol, 1,1-Ethanediol, 1,4-Cyclohexanediol, 1,2,3-Propanetriol, 1-(1-hydroxyethyl)-1-methylcyclopropane, 2-(1-methylcyclopropyl)ethanol, 2-(2-hydroxyethyl)-2-methylcyclopropanol, 3-(2-hydroxyethyl)-3-methyl-1,2-cyclopropanediol, 1,2-Di(hydroxymethyl)cyclohexane and 2-(hydroxymethyl)-1,3-propanediol, and mixtures thereof), and acids at about 0.01% w/w to 10% w/w, or about 0.1% w/w to 3% w/w (e.g. formic acid, acetic, nitric acid, hydrochloric acid, sulphuric acid, benzoic acid, hydrobromic acid, hydroiodic acid, perchloric acid, chloric acid, oxalic acid, sulfurous acid, hydrogen sulfate ion, phosphoric acid, nitrous acid, hydrofluoric acid, methanoic acid, benzoic acid, acetic acid, formic acid, lactic acid, acetic acid, formic acid, citric acid, oxalic acid, uric acid, malic acid, tartaric acid, and mixtures thereof).

Preferably, the mixing of the two or more cationic antimicrobials is carried out in at least two distinct steps. For instance, a first cationic antimicrobial is mixed into the clay solution for a first period of time and, thereafter, the second cationic antimicrobial is mixed into the clay solution (comprising the mixed 1^{st} antimicrobial) for a second period of time.

Biologically active or non-biologically compounds may be added to the clay solution addition to the antimicrobial(s). In embodiments, the biologically active or non-biologically compound(s) are added separately to the antimicrobial(s) (e.g. before or after).

In a preferred embodiment, the mixing of the two or more cationic antimicrobials (and/or cationic antimicrobial+biologically active or non-biologically compound(s)) is carried out in separate steps, wherein the first compound to be added to clay is “smaller” than the second compound, wherein “smaller” is based on a comparative size of the cations in the two compounds, rather than the total size or molecular weight of compounds.

The mixing conditions may vary according different factors including, but not limited to, temperature, concentrations of the antimicrobial(s), type of antimicrobials, type(s) of clay(s), lattice space of the clay(s), etc. For instance, generally the cation exchange process will be faster a higher temperature (e.g. 30° C. vs 4° C.), but too high temperature (e.g. >70° C.) may not be advisable because high temperatures may be detrimental to the integrity of the antimicrobial(s) or of the clay.

In embodiments mixing is between a few minutes (e.g. 1, 5, 30, 45 or 60 minutes) to days (e.g. 1, 2, 3, 4 or 5 days). In embodiments, the first antimicrobial is mixed for about 1 hour with the clay, before adding the at least second antimicrobial. The second antimicrobial (or more antimicrobial) is then mixed with the clay for a few more hours (e.g. 5, 12, 20, 23 hours).

The amount or concentration of antimicrobials may vary according to particular mixing conditions or desired commercial use(s) and/or desired potency of the nanoclay. Typically, less potent antimicrobial nanoclays can be synthesized by having a lower molar ratio of antimicrobials to clay. For example, a 0.5:1 ratio antimicrobials:phyllosilicate would provide less antimicrobial efficacy than a 1:1 ratio or a 2:1 molar ratio but will still be suitable for many uses and under many circumstances. In embodiments, the antimicrobials are added to the phyllosilicate clay at a molar ratio of about 0.1 to about 5 moles total of antimicrobial agents per mole of phyllosilicate clay. In embodiments, the ratio antimicrobials:phyllosilicate clay is about 3:1 or 2:1 or 1:1 or 0.5:1. In preferred embodiment, the ratio is 2:0, wherein the ratio for the antimicrobials is divided substantially equally between each of the antimicrobials, for instance, for two antimicrobials a ratio of 1 for each of antimicrobial and 1 for the clay (i.e. 1:1:1), for three antimicrobials a ratio of about 0.66 for each of antimicrobial and 1 for the clay (0.66; 0.66:0.66:1), for four antimicrobials a ratio of about 0.5 for each of antimicrobial and 1 for the clay (0.5; 0.5:0.5; 0.5:1), etc. Those skill in the art can also appreciate that adding excess antimicrobials may be useful to make sure the reaction solution has enough antimicrobial cations to exchange with the cations (Na⁺) in the nanoclay.

For instance the final mixture of the antimicrobials may be between 1% w/w to 99% w/w of the first antimicrobial agent and 99% w/w to 1% w/w of the second antimicrobial agent (adding to 100%). If the antimicrobial nanoclay comprises three antimicrobials, then the final mixture of the antimicrobials may be between 1% w/w to 98% w/w of the first antimicrobial agent, 1% w/w to 98% w/w of the second antimicrobial agent and 1% w/w to 98% w/w of the third antimicrobial agent (adding to 100%), and so on if there is four or more antimicrobials. As will be appreciated by one of skill in the art, this represents the mix for the exchange reaction. The ratio in the final product will be a bit different.

In one embodiment, the nanoclay comprises a combination of two different cationic antimicrobial agents, for example, benzalkonium chloride and silver nitrate each approximately at a molar ratio 1:1 in the nanoclay (final 1:1:1). Such antimicrobial nanoclay may provide a robust antimicrobial additive material to eradicate multi-drug resistance bacteria and spores found in hospital and healthcare settings.

In another particular embodiment, the nanoclay comprises equal ratios of quaternary ammonium compound and silver ions. Such combination may exert superior bactericidal effects on pathogens such as Enterococcus faecium, Staphylococcus aureus, Klebsiella pneumoniae, Acinetobacter baumannii, Pseudomonas aeruginosa, Enterobacter spp, Clostridium difficile and Candida albicans, owing to the multiple mode of actions of the two different antimicrobials.

It may also be envisioned to reduce the concentration of one of the antimicrobial agents, for example, silver (e.g. from 10% to 1%) since, thanks to the presence of the other antimicrobial, the antimicrobial nanoclay could still be effectively used against semi-pathogenic or wild type susceptible pathogens such as E. coli, S. aureus, etc. both in hospitals and community settings.

The mixture obtained after mixing the antimicrobials with the clay can then be further treated to obtain a concentrated paste or a powder. For instance, the mixture can be decanted, centrifuged, filter, dried, etc.

In embodiments the mixture it left to settle for 1 hour or more. In embodiments, the mixture is centrifuged (e.g. about 5000-about 10 000 RPM) for 5 to 20 min, or more. In embodiments, the mixture is filtered pressed (e.g. through a mesh of about 100 to about 500 micron), for separating the solids out of the liquid solution. In embodiments, any of these steps are repeated a few times and water is added in between for rinsing to obtain a nanoclay paste that is substantially free of unbound antimicrobials.

If desired the nanoclay paste can be dried to a power form (e.g. using a freeze dryer, oven, flatbed dryer, fluidized bed dryer). The obtain powder may subsequently be grinded to obtain smaller particles of a desired size (e.g. ball bill, two roll mill, hammer mill).

### Commercial Applications of the Nanoclays

A large variety of articles of manufacture may benefit from incorporating an antimicrobial nanoclay as defined herein. These include, but are not limited to, paints, polymers, ceramics, filters, foams, fibers, textiles, leather, paper and cellulose. For instance, an antimicrobial nanoclay as defined herein articles can be incorporated into these articles to confer them with desired antimicrobial activity.

For example, a wide range of paint products could be integrated with the antimicrobial nanoclay such as for example, but by no means limited to, primers, emulsion paints, varnish, wood stain, lacquer, enamel paint, roof coating, powder coating, inks, anti-graffiti coatings, anti-climb paint, anti-fouling paint, insulating paint, anti-slip paint and luminous paint.

Additionally, polymers, especially thermoplastics, thermosets and elastomers could be infused with the antimicrobial nanoclay of the invention. For example, wide range of polymers such as, but by no means limited to, biopolymers, inorganic polymers, organic polymers, conductive polymers, copolymers, fluoropolymers, phenolic resins, polyketones, polyesters, polyolefins (polyalkene), rubber, silicone, silicone rubber, superabsorbent polymers, synthetic rubber, vinyl polymers and the like could be infused and/or coated with the antimicrobial nanoclay of the invention.

As will be appreciated by one of skill in the art, preparation of antimicrobial nanocomposites for industrial quantities may require modified synthesis protocols and equipment. For example, the drying of antimicrobial nanoclay(s) may require a fluidized bed dryer to handle large quantities, whereas a freeze-dryer may be used in lab settings. As another example, while a centrifuge is used to separate precipitates on a laboratory scale, a filter-press may be used for larger scale production. Similar types of modifications will be readily apparent to one of skill in the art.

Exfoliation of the antimicrobial nanoclay(s) defined herein into a polymer/paint matrix may be advantageous to achieve enhanced antimicrobial efficacies. Better exfoliation of nanocomposite can, not only increases their mechanical properties, but also highly influences the distribution of antimicrobial scaffolds throughout the polymer matrix. Exfoliation of the nanoclays may also avoid the formation of large agglomerates of particles that could have a negative effect on the integrity of the nanoclay layers. In embodiments, during the mixing (shear mixing or sonication), these clay layers are separated and spread evenly onto the polymer (paint) paint to form exfoliated composites. For example, assume that each nanolayer is 1 nm thick and has a 1-micron average diameter. Every layer consists of numerous antimicrobial agents (for example both Ag and benzalkonium ions). Hence, in a small coated area, there are millions of antimicrobial ions present compared to thousands to few hundred thousand when larger micron-sized antimicrobials are used. Because of this, nanoclay-based antimicrobials would exert better antimicrobial activity and for a longer duration. Even if an antimicrobial is spent in killing bacteria, there are always unspent antimicrobials in adjacent layers. Furthermore, once an exposed surface wears off, there are still thousands of nanolayers beneath which are impregnated with antimicrobial agents and can form a new exposed surface, thereby making sure that the antimicrobial efficacy of the coating lasts as long as the integrity of the paint is maintained. It is within the skill of those in the art to identify suitable solvents and techniques for exfoliation or even diffusion/distribution into the product matrix, depending on the desired end product.

In some embodiments, visual and/or physical observation of the consistency and/or homogeneity of the end product may be tested to determine the degree of mixing, that is, to determine if additional mixing is required.

It is important to note that a surface painted with a paint product or coated with a polymer product comprising the antimicrobial nanoclay of the invention will have thousands of layers of clay sheets, based on the fact that each sheet has an approximate thickness of 5-10 micrometers. This, of course, will be higher depending on the number of coatings on a given surface, as more coatings would lead to more thickness, and therefore a greater number of nanolayers.

Also, the invention encompasses the uses of an antimicrobial nanoclay as defined herein for providing antimicrobial activity to a surface. In embodiments, the antimicrobial nanoclay could be applied in a substantially liquid format, for example, as a spray or by a brush, such that the antimicrobial nanoclay dries on the applied surface. For instance, the antimicrobial nanoclay may be applied to contacted surfaces in a care setting, for example, in a hospital or care home or other similar facility. Many different types of surface may benefit from being contacted with the antimicrobial nanoclay of the invention, particularly surfaces with which patients or other individuals may touch with their hands which may in turn result in an infection for the individual and/or spread an infection to other individuals. Examples of such surfaces include, but are not limited to, furniture such as chairs, walls, bedside tables and the like, partition curtains, hand rails, doors and doorknobs, medical equipment, structural elements within a care facility relating to the building itself and/or its construction such as walls, floors, elevators and the like, as well as parts of the heating and air system, for example, air vents, air filters and the like, thereby severely curtailing the spread of infective agents such as bacteria. In one particular embodiment the antimicrobial nanoclay material is used in vents and/or air filters for avoiding spread of airborne microbes, thereby preventing issues such as “sick building syndrome” from developing.

Performance of the antimicrobial nanoclay(s), and/or antimicrobial activity of a surface contacted with same may be assessed with any suitable technique or device including, but not limited to, antimicrobial efficacy testing, or using a handheld FTIR spectroscope. Specifically, regarding the use of a handheld FTIR spectroscope as long as antimicrobials are present in the coated surface, they will show distinct peaks in the FTIR spectra. Based on the presence and intensity of these peaks, the presence and quantity of respective antimicrobials in the coating can be estimated.

A further related aspect of the invention relates to kits. The kits of the invention may be useful for the practice of the methods of the invention, particularly for providing antimicrobial activity to a surface.

A kit of the invention may comprise one or more of the following components:
an antimicrobial nanoclay as defined herein; and
at least one additional component including, but not limited to, a user manual or instructions, a spray bottle, a mixing bottle, pen(s), marking sheets, boxes, holders, wipes, and cleaning solutions.

In the kit, the antimicrobial nanoclay may be provided in a concentrated paste or in a powder form. The antimicrobial nanoclay may also be formulated as a concentrate for a dilution prior to use.

In some embodiments, there may be two, three or four different antimicrobials in the nanoclay. In other embodiments, there may be two or three different antimicrobials in the nanoclay, as discussed herein.

Those skilled in the art will recognize, or be able to ascertain, using no more than routine experimentation, numerous equivalents to the specific procedures, embodiments, claims, and examples described herein. Such equivalents are considered to be within the scope of this invention, and covered by the claims appended hereto. The invention is further illustrated by the following examples, which should not be construed as further or specifically limiting.

### EXAMPLES

A series of experiments were carried out for demonstrating feasibility and antimicrobial activity of antimicrobial nanoclays as defined herein. Unless stated otherwise, all the ingredients such as nanoclay and antimicrobials are added in grams per liter. Bold number in parenthesis (#) refers to the material listed in Tables 1-3.

### Materials and Methods:

Hexadecylpyridinium chloride, Polymyxin B and Niclosamide were purchased from AK Scientific Inc. Copper sulfate and Benzyldimethyl tetradecylammonium chloride were purchased from Fischer Scientific. All the others used chemicals were purchased from Sigma-Aldrich unless otherwise specified. The XRD was performed using Rigaku Ultima-IV powder x-ray diffractometer with a Cu source and scintillation detector. X-rays were generated at 40 kV and 44 nA. The scan range is 3-40 deg 20, the step size is 0.02 deg and count time is 1 sec. The Scanning Electron Microscope (JEOL JSM 7100F, equipped with a field emission source and operating at 15 kV) combined with Energy Dispersive X-Ray spectroscopy (EDX) was used to observe the morphology and composition of the modified bentonite particles and coated surfaces.

### Example 1—Mono Cationic Exchange

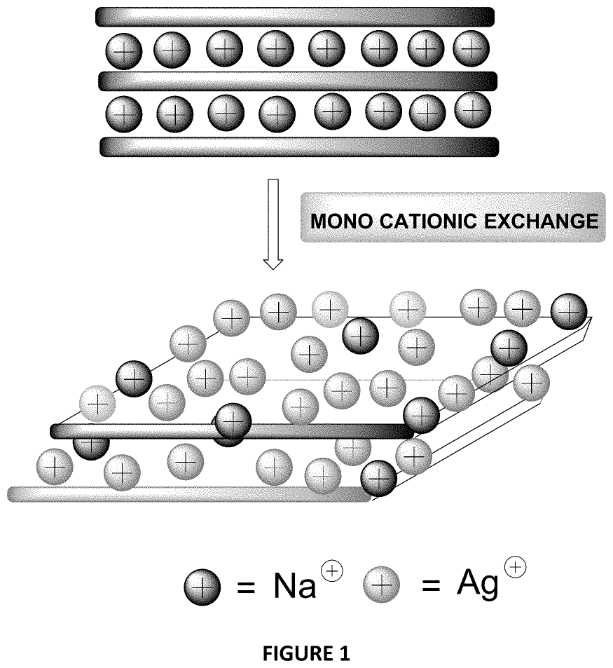

Initially, silver ions have been incorporated into bentonite nanoclay (BN-Ag, material 1) via cationic exchange process (FIG. 1). Similarly, benzalkonium chloride (BAC), a versatile quaternary ammonium compound (material 6) had been incorporated into bentonite nanoclay and further characterized using sophisticated analytical instruments. Similarly, a series of antimicrobials were incorporated into nanoclay system via cationic exchange process. They include, bentonite with different metal ions such as copper (BN-Cu, material 3), gallium (BN-Ga, material 4), a zinc complex (BN-ZnPY, material 5), quaternary ammonium compounds (QACs) (materials 6-8), antimicrobials/antibiotics (materials 9-15), aminopolycarboxylic acid (material 16) (Table 1).

TABLE 1
Mono cationic exchange nanomaterials

| | Material |
|---|---|
| Compound | number |
| Bentonite-Silver (BN-Ag) | 1 |
| Halloysite-Silver (HS-Ag) | 2 |
| Bentonite-Copper (BN-Cu) | 3 |
| Bentonite-Gallium (BN-Ga) | 4 |
| Bentonite-Zinc pyrithione (BN-ZnPY) | 5 |
| Bentonite-Benzalkonium chloride (BN-BAC) | 6 |
| Bentonite-Benzyldimethylhexadecylammonium chloride (BN- | 7 |
| BDMHAC) | |
| Bentonite-Benzyldimethyltetradecylammonium chloride (BN- | 8 |
| BDMTAC) | |
| Bentonite-Hexadecylpyridinium chloride (BN-HDPYC) | 9 |
| Surface modified clay- Hexadecylpyridinium chloride (SMC- | 10 |
| HDPYC) | |
| Halloysite- Hexadecylpyridinium chloride (HS-HDPYC) | 11 |
| Bentonite-Chlorohexidine (BN-CHL) | 12 |
| Bentonite-Neomycin (BN-NM) | 13 |
| Bentonite-Polymyxin-E (BN-PMX) | 14 |
| Bentonite-Niclosamide (BN-NCL) | 15 |
| Bentonite-Ethylenediaminetetraacetic acid (BN-EDTA) | 16 |

The experimental design involves the synthesis of antimicrobial-clay hybrids. The following procedure has been undertaken: bentonite clay was first dispersed in deionized water under stirring about 200 rpm for one hour. The pH of the solution was brought to 4-5 using aqueous nitric acid. A pre-dissolved stoichiometric amount of multiple antimicrobials (or single antimicrobial, depending on the sample type) were slowly added to the clay suspension at room temperature. The concentrations of antimicrobials used are 2.0 CEC (cation exchange capacity) and 1.0 CEC of the bentonite clay, respectively. The reaction mixtures were stirred for 24 h at room temperature. The mixtures then have been centrifuged, rinsed with water for three times and put into freeze drying for one day. Freeze drying is a low temperature dehydration process, which involves freezing the product, lowering pressure, then removing the ice by sublimation. All the antimicrobial incorporated nanoclay products were then dried at room temperature and stored in plastic vials. We have used various combinations of antimicrobials to incorporate onto the bentonite and other nanoclays by the cation exchange process.

As will be appreciated by one of skill in the art, the CEC ratio of 1:2 between the bentonite and antimicrobials was used initially so as to make sure that as much of the antimicrobial agents are getting into the nanoclay. However, the process will work the same for any CEC ratios. As such, any suitable ratio between nanoclays such as bentonite and the antimicrobial agents may be used, which can be determined through routine experimentation and are within the scope of the invention.

### Example 2—Dual Cationic Exchange

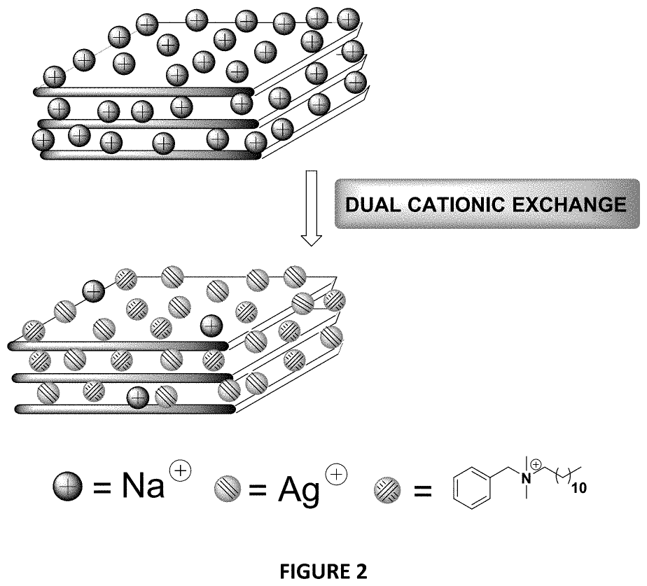

To investigate the dual cationic exchange process, both silver and benzalkonium chlorides (BAC) were incorporated into bentonite nanoclay system via cationic exchange process (FIG. 2), which resulted into a new nanoclay material (material 22) that harbors electrostatically inked silver and benzalkonium ionic molecules. In dual cationic exchange process two different cationic entities are intercalated between clay layers. Several combinations of dual antimicrobials have been incorporated on to bentonite, halloysite, montmorillonite and surface modified montmorillonite (see Table 2).

TABLE 2
Dual cationic exchange nanomaterials

| | Material |
|---|---|
| Compound | number |
| Bentonite-Silver-Copper (BN-Ag—Cu) | 17 |
| Bentonite-Silver-Gallium (BN-Ag—Ga) | 18 |
| Bentonite-Copper-Gallium (BN-Cu—Ga) | 19 |
| Bentonite-Copper-Zinc pyrithione (BN-Cu-ZnPY) | 20 |
| Bentonite-Silver-Zinc pyrithione (BN-Ag-ZnPY) | 21 |
| Bentonite-Silver-Benzalkonium chloride (BN-Ag-BAC) | 22 |
| Bentonite-Copper-Benzalkonium chloride (BN-Cu-BAC) | 23 |
| Bentonite- Silver-Benzyldimethylhexadecylammonium | 24 |
| chloride (BN-Ag-BDMHAC) | |
| Bentonite-Silver-Benzyldimethyltetradecylammonium | 25 |
| chloride (BN-Ag-BDMTAC) | |
| Bentonite-Copper-Benzyldimethylhexadecylammonium | 26 |
| chloride (BN-Cu-BDMHAC) | |
| Bentonite-Copper-Benzyldimethyltetradecylammonium | 27 |
| chloride (BN-Cu-BDMTAC) | |
| Bentonite-Benzalkonium chloride- | 28 |
| Benzyldimethylhexadecylammonium chloride (BN-BAC- | |
| BDMHAC) | |
| Bentonite-Neomycin-Chlorohexidine (BN-NM-CHL) | 29 |
| Surface modified clay-Neomycin- Chlorohexidine | 30 |
| (SMC-NM-CHL) | |
| Montmorillonite- Neomycin-Chlorohexidine (MMT-NM-CHL) | 31 |
| Halloysite- Neomycin-Chlorohexidine (HS-NM-CHL) | 32 |
| Bentonite-Polymyxin E-Niclosamide (BN-PMX-NCL) | 33 |
| Bentonite-Silver- Ethylenediaminetetraacetic acid | 34 |
| (BN-Ag-EDTA) | |
| Bentonite- Benzalkonium chloride- | 35 |
| Ethylenediaminetetraacetic acid (BN-BAC-EDTA) | |

### Example 3—Triple Cationic Exchange

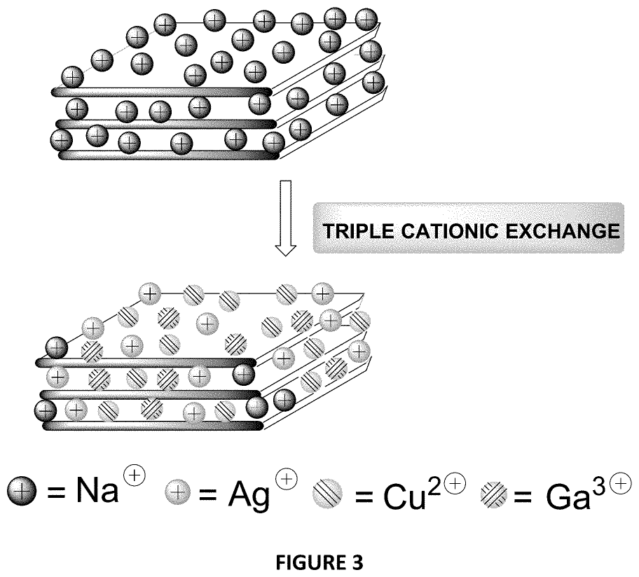

A triple antimicrobial combination was accomplished in bentonite nanoclay system. Three various metal/antimicrobials were incorporated into bentonite via triple cationic exchange (FIG. 3). For example, bentonite-triple metal (material 36), bentonite-metal-quaternary ammonium compound-zinc complex (materials 37-38), bentonite-triple quaternary ammonium compound (material 39) and bentonite-antibiotics-EDTA (material 40) were successfully synthesized and characterized via spectroscopic methods (Table 3).

TABLE 3
Triple cationic exchange nanomaterials

| | Material |
|---|---|
| Compound | number |
| Bentonite-Silver-Copper-Gallium (BN-Ag—Cu—Ga) | 36 |
| Bentonite-Silver-Benzalkonium chloride- Zinc pyrithione | 37 |
| (BN-Ag-BAC-ZnPY) | |
| Bentonite- Silver-Benzyldimethylhexadecylammonium | 38 |
| chloride- Zinc pyrithione (BN-Ag-BDMHAC-ZnPY) | |
| Bentonite-Benzalkonium chloride- | 39 |
| Benzyldimethylhexadecylammonium chloride- | |
| Benzyldimethyltetradecylammonium chloride | |
| (BN-BAC-BDMHAC-BDMTAC) | |
| Bentonite-Neomycin-Chlorohexidine- | 40 |
| Ethylenediaminetetraacetic acid (BN-NM-CHL-EDTA) | |

Antimicrobial surface testing: Finally, antimicrobial nanomaterial obtained by dual cationic exchange process was subjected to “exfoliation” process in an appropriate polymeric binding material, in order to develop antimicrobial polymer nanocomposites. These polymer nanocomposites eventually could be deployed as antimicrobial coating materials. Exfoliation could be defined as process of dispersion of the nanoclay particles in the polymer matrix. Exfoliation provides enhanced physio and biological properties to the nanoclay based polymer composites. Silver-QAC containing bentonite was exfoliated in an acrylic solvent via sonication. The resulting polymeric solution was thinly coated over a 5×5 cm² acrylic sheet and dried overnight (material 43). The coated samples were tested to evaluate antimicrobial properties by following standard JIS Z 2801:2000 (JIS Z 2801: 2000 Antimicrobial products-Test for antimicrobial activity and efficacy. (2000)).

Two major steps are involved in clay particle dispersion in polymers-intercalation and exfoliation. In the intercalation step, the spacing between individual clay layers, called d-spacing, increases from their intrinsic values as polymer chains or monomer molecules diffuse into the clay galleries, facilitated by the treatment of clay particles with organic modifiers, such as hydrophobic quaternary alkylammonium ions. In exfoliation, the individual clay particles are separated from the intercalated tactoids and are dispersed in the matrix polymer with no apparent interparticle interactions. In other words, in nanocomposites, exfoliation is the state when the clay layer spacing increases to the point where the attraction between the clay particles no longer exists. There are many different dispersion techniques lead to varying degrees of exfoliation. Dispersion methods such as sonication, magnetic stirring and “thicky” mixing, each achieve reasonable exfoliation of the nanoclay particles in the polymer. Other suitable dispersion methods will be readily apparent to one of skill in the art and are within the scope of the invention. For example, three roll milling is an effective dispersion method that disentangles the nanoclay particles and increases the clay galleries that enable the polymer molecules to penetrate.

After the cationic exchange process, the end user must disperse or exfoliate the antimicrobial nanomaterial in an appropriate solvent (depending on the end application) and apply to achieve maximum efficacy.

### Example 5—Incorporation of Dual Antimicrobials

### Incorporation of Metal and Quaternary Ammonium Compound (QAC)

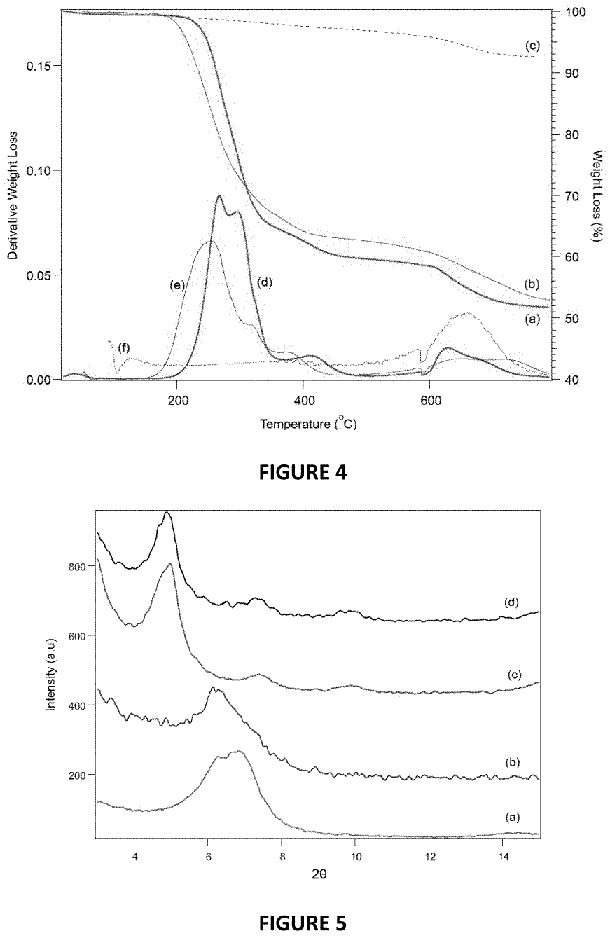

FIG. 4 shows the weight loss and derivative weight loss thermograms of bentonite incorporated with Ag, BDMHAC (Benzyldimethyl hexadecyl ammonium chloride) and both. The weight losses of BDMHAC incorporated bentonite and Ag incorporate bentonite are 52.9 and 92.53%. The derivative weight loss peaks for the BN-Ag-BDMHAC (material 22) sample has the peaks of bentonite+Ag and bentonite+BAC, indicating that both Ag and BKC are present in the sample.

The enhancement of the value d(001) indicates intercalation of alkylammonium cations into the bentonite (FIG. 5). The d-spacing of bentonite is 1.30 nm, which increases to 1.40, 1.78 and 1.83 nm when treated with Ag, BDMHAC and Ag-BDMHAC.

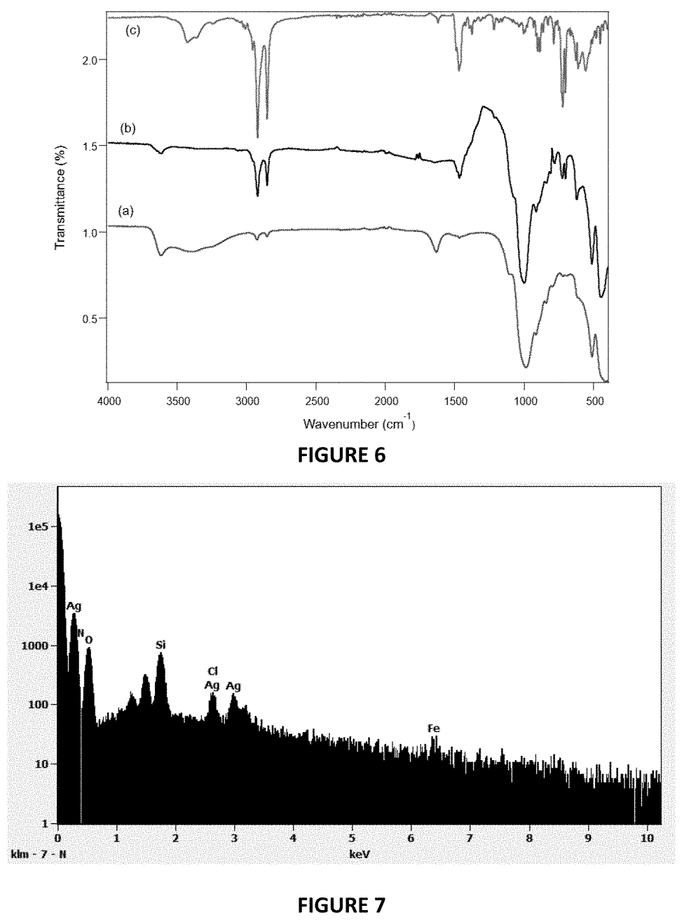

FIG. 6a shows the FTIR spectrum of BDMHAC. The stretching vibrations of the C—H bonds occurring in the 2850-2950 cm⁻¹ region reflect C16 chains of BDMHAC. The bending vibrations of C—H fragments from BDMHAC appear at 1467 cm¹ (Navrátilová, Z et al. V. Sorption of alkylammonium cations on montmorillonite. Acta Geodyn. Geomater. 4, 59-65 (2007)). The presence of all these peaks in the BN-BDMHAC sample indicate the alkylammonium cations are present in the modified bentonite.

FIG. 7 shows the EDX spectrum of BN-Ag-BDMHAC (material 24) sample. Since the FTIR does not show the presence of Ag in the sample, we have used EDX to verify the presence of Ag in the sample. This plot clearly shows the presence of Ag in the sample. While the FTIR shows the presence of BDMHAC, EDX confirms the presence of Ag. Hence, this demonstrated that our unique cation exchange process method; we can incorporate two different classes of antibacterial (metal and QAC) onto the bentonite.

Biological Data: In order to demonstrate combination “synergy effect” in nanoclay system, a series of antimicrobial evaluation experiments were conducted with various antimicrobial metals/compounds. The antimicrobial activity (minimum inhibitory concentration, MIC) was evaluated by employing microbroth dilution method to determine the lowest concentration of the assayed antimicrobial additive material. Individual antimicrobials when incorporated into the nanoclay (bentonite) system displayed no or low activities against clinically relevant bacteria.

For example, bentonite-silver (material 1), quaternary ammonium salts incorporated bentonite (materials 6 and 7) showed no activity (MIC ranging from 32-1024 μg/mL) against both Gram-negative and Gram-positive bacteria except on MRSA (SA001). Then the combined synergy efficacy of antimicrobial metal ion (silver) and quaternary ammonium salts incorporated into the bentonite nanoclay (materials 22 and 24), via “dual metal/organic cation exchange” was investigated on the drug resistant pathogens. Antimicrobial activities of BN-Ag-BAC (material\ 22) and BN-Ag-BDMHAC (material 24) clearly demonstrates the “synergy effect”. Both the samples displayed superior antimicrobial activities (MIC range 8-32 μg/mL) against drug-resistant Gram-negative and Gram-positive bacteria (Tables 4 and 5).

TABLE 4
Dual cationic exchange nanomaterial with carried concentrations

| | Material |
|---|---|
| Compound | number |
| Bentonite- Silver- Benzalkonium chloride (BN-Ag-BAC) | 41 |
| in the % of 1:0.1:0.9 respectively | |
| Bentonite-Silver- Benzalkonium chloride (BN-Ag-BAC) | 42 |
| in the % of 1:0.5:0.5 respectively | |
| Bentonite- Silver- Benzalkonium chloride (BN-Ag-BAC) | 43 |
| in the % of 1:0.5:1.5 respectively | |

To validate combination synergy effect, equal weight percentage (1:1) of bentonite-silver (material 1) and bentonite-benzalkonium chloride (material 6) were physically ground together. The resulting physically mixed nanomaterial (material 44) was subjected to antimicrobial efficacy testing. This material did not show antibacterial activity against an array of various bacterial isolates. For example, material 44 showed MIC of 256 μg/mL against clinically relevant P. aeruginosa (PA07). This experiment emphasizes the importance of “chemical” combination (inside the nanoclay system) of antimicrobial materials than the mere physical combination of two individual antimicrobial nanomaterials.

It is worth to note that, varied concentrations of dual antimicrobial nanoclay systems such as materials 41, 42 and 43 with BN-Ag-BAC in the order of 1:0.1:0.9, 1:0.5:0.5 and 1:0.5:1.5 ratios respectively proved to be less effective when compared to material 22 with 1:1:1 ratio of bentonite and antimicrobials (Table 5). As discussed herein, in some embodiments, lower efficacy materials may be desirable for certain applications.

A similar study for BN-Ag (material 1), BN-BDMHAC (material 7) and BN-Ag-BDMHAC (material 45) samples are shown in Table 6.

TABLE 5
MIC (μg/mL) of BN-Ag, BN-BAC and BN-Ag-BAC with varied concentrations

| | | | | | BN-Ag- | BN-Ag- | BN-Ag- | BN-Ag- | |
|---|---|---|---|---|---|---|---|---|---|
| | | | | | BAC | BAC | BAC | BAC | Physical |
| Bacterial | Only | Only | BN-Ag | BN-BAC | (1:0.1:0.9) | (1:0.5:0.5) | (1:0.5:1.5) | (1:1:1) | mixing of 1 + 6 |
| strain | Ag | BAC | (material 1) | (material 6) | (material 41) | (material 42) | (material 43) | (material 22) | (material 44) |
| P. aeruginosa | 2 | 32 | 128 | >1024 | 512 | 256 | 256 | 16 | 256 |
| (PA07) | | | | | | | | | |
| E. cloacae | 32 | 8 | 256 | 512 | 128 | 128 | 64 | 32 | 128 |
| (EB5) | | | | | | | | | |
| A. baumannii | 8 | 2 | 256 | 128 | 64 | 256 | 32 | 16 | 256 |
| (AB034) | | | | | | | | | |
| MRSA | 8 | 1 | 256 | 16 | 16 | 32 | 16 | 8 | 64 |
| (SA001) | | | | | | | | | |
| E. coli (K12) | 2 | 16 | 128 | 512 | 256 | 128 | 128 | 8 | 256 |
| K. pneumoniae | 8 | 4 | 256 | 128 | 64 | 64 | 32 | 16 | 128 |
| (KP001) | | | | | | | | | |

TABLE 6
MIC (μg/mL) of BN-Ag, BN-BDMHAC and BN-Ag-BDMHAC

| | | | | Physical mixing of |
|---|---|---|---|---|
| | BN-Ag | BN-BDMHAC | BN-Ag-BDMHAC | 1 + 7 |
| Bacterial strain | (material 1) | (material 7) | (material 24) | (material 45) |
| P. aeruginosa (PA07) | 128 | 256 | 32 | 256 |
| E. cloacae (EB5) | 256 | 1024 | 32 | >1024 |
| A. baumannii (AB034) | 256 | 32 | 16 | 256 |
| MRSA (SA001) | 256 | 8 | 2 | 128 |
| E. coli (K12) | 128 | 512 | 16 | 256 |
| K. pneumoniae (KP001) | 256 | 32 | 16 | 64 |

### Example 6—Bentonite+Neomycin+Chlorohexidine (BN-NM-CHL)

In this experiment, we have shown that two organic antibacterials such as chlorohexidine (antiseptic) and neomycin (antibiotic) could be incorporated onto the bentonite (material 29). Balanchard et al. have reported that neomycin sulfate enhances the antimicrobial activity of Mupirocin-based antibacterial ointments. They found the improved antimicrobial activity of neomycin and mupirocin in ointment formulations and reduced S. aureus bacterial burden in wound site infections (Blanchard, C. et al. Neomycin Sulfate Improves the Antimicrobial Activity of Mupirocin-Based Antibacterial Ointments. Antimicrob Agents Chemother. 60, 862-872 (2016)). However, there is no literature found in which both chlorohexidine and neomycin have been incorporated onto the nanoclay.

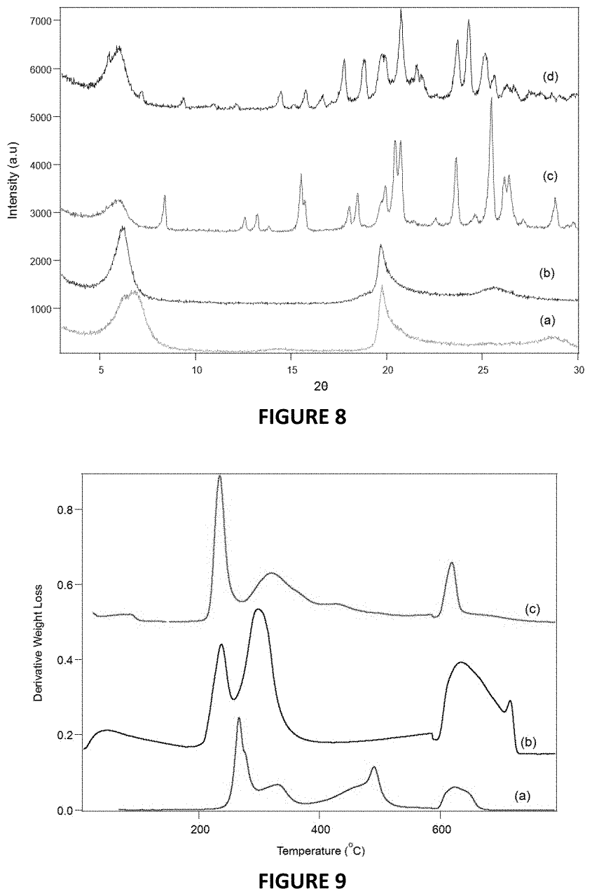

FIG. 8 shows the XRD data of bentonite, BN-NM, BN-CHL and BN-CHL-NM samples. The interlayer d-spacing of the bentonite can be calculated using the first 2θ peak (based on Bragg's law, 2d sin θ=nλ, where d-clay spacing, λ-wavelength of the X-ray). The downward shift in the 2θ peak indicates the increase of d-spacing of nanoclay. For the pure nanoclay, the 2θ peak is at 6.82, which decreases to 4.82 once the antimicrobial is incorporated. As a result, the d-spacing of nanoclay increases from 1.40 nm to 1.83 nm after the incorporation of AM. The increase in d-spacing is due to the fact that the size of the AM is much higher than the Na⁺ ions that it replaces in the clay.

It could be seen that the TGA derivative peak of the material 29 (FIG. 9) shares some common decomposition patterns of the antimicrobials it is made of. This indicates the presence of both antimicrobials in the mixture, and thus, high affinity towards the clay.

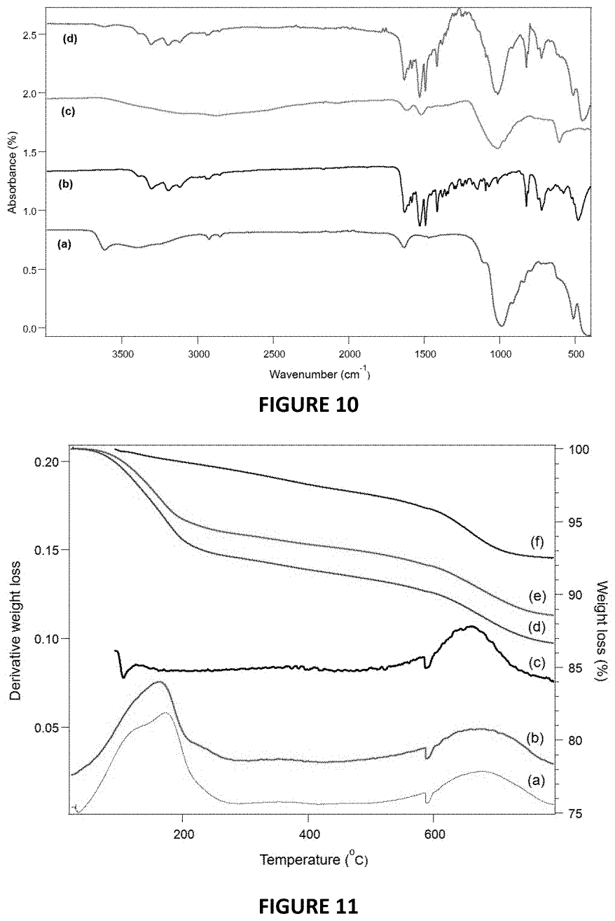

Fourier Transform Infrared (FTIR) spectroscopy has been used to analyze the surface functionality of materials. FIG. 10a shows the FTIR data of unmodified bentonite. The 3621 cm⁻¹ and 916 cm⁻¹ bands are typical of dioctahedral smectites. The 3612 cm⁻¹ and 1632 cm⁻¹ bands corresponded to OH frequencies of the water molecule. The band at 991 attributes to Si—O, 513 & 454 attribute to low frequency SiO, 1105 attribute to high frequency SiO, 1632 to OH bending vibration of water, and 3612 to OH stretching vibration zone from Al—Al—OH (Alabarse, F. G., et al. In-situ FTIR analyses of bentonite under high-pressure. Appl. Clay Sci. 51, 202-208 (2011) and Zhirong, L., et al. Molecular and Biomolecular Spectroscopy FT-IR and XRD analysis of natural Na-bentonite and Cu (II)-loaded Na-bentonite. Spectrochim. Acta Part A Mol. Biomol. Spectrosc. 79, 1013-1016 (2011)). The bands at 1013 and 1519 are from C—O stretching and C═O stretching of neomycin (FIG. 10c). The bands at 3308, 1628, 1579, 1090 and 726 cm⁻¹ are due to —NH, C═O aromatic, N—H bend, aliphatic C—N and C—Cl of chlorohexidine (FIG. 10b) (Lohar, R. J., et al. FT-IR spectrophotometric analysis of chlorhexidine hydrochloride and its pharmaceutical. World J. Pharm. Pharm. Sci. 5, 1702-1705 (2016)).

FTIR results indicate that material 29 spectrum lies in between both of antimicrobials it is made of (FIG. 10d). In general, material 29 has the same functional groups that are present in bentonite and both antimicrobials.

Single antimicrobial containing bentonite materials, Bentonite-Chlorohexidine (BN-CHL, material 12) and Bentonite-Neomycin (BN-NM, material 13) showed primarily no or moderate activity against all the strains tested (Table 7). For example, individually incorporated antimicrobials into bentonite, materials 12 and 13 displayed MIC 64 and >1024 μg/mL respectively against clinically relevant P. aeruginosa (PA07). In contrast, combined integration of neomycin and chlorhexidine into bentonite system (material 29) resulted in MIC of 2 μg/mL (524 to 64 folds higher antimicrobial efficacy than individual components) against P. aeruginosa (PA07).

TABLE 7
Antimicrobial activities in MIC (μg/mL) of BN-CHL (material 12), BN-NM (material
14) and BN-NM-CHL (material 29) against a series of clinically relevant bacteria

| | | | | | | BN-NM- |
|---|---|---|---|---|---|---|
| | Neomycin | Chlorhexidine | Bentonite | BN-CHL | BN-NM | CHL |
| | (NM) | (CHL) | (BN) | (material 12) | (material 13) | (material 29) |
| P. aeruginosa | 128 | 16 | >2048 | 64 | >1024 | 2 |
| (PA07) | | | | | | |
| K. pneumoniae | 2 | <0.5 | >2048 | <0.5 | 1024 | 0.5 |
| (KP001) | | | | | | |
| A. baumannii | 8 | 256 | >2048 | 512 | >1024 | 16 |
| (AB30) | | | | | | |
| E. coli | 2 | 2 | >2048 | 2 | >1024 | 2 |
| (E524) | | | | | | |
| E. cloacae | 4 | 8 | >2048 | 4 | >1024 | 2 |
| (EB5) | | | | | | |
| MRSA | 4 | 8 | >2048 | 4 | >1024 | 2 |
| (SA004) | | | | | | |

### Example 7—Incorporation of Two Metals onto Bentonite

In these experiments, we have incorporated individual metals of Ga, Ag or Cu (using Ga(NO₃)₃, AgNO₃, and CuSO₄) or combinations of metals using the above-mentioned cation exchange process. The CEC ratio between the bentonite and antimicrobials were kept at 1:2.

The residue of BN-Ag (material 1) sample has increased to 92.5 from 82% of bentonite alone, indicating that more thermally stable Ag has been incorporated on to the bentonite (FIG. 11). In the case of BN-Cu (material 3) sample, the residue is 86.66%. The residue to both Cu and Ag incorporated bentonite (material 17) is 88.66%, which lies between that of the individual metal incorporated bentonite indicating that both might be present in the sample. All these increases in residue value for these samples, indicating the incorporation of metals onto the d-layer spacing of bentonite.

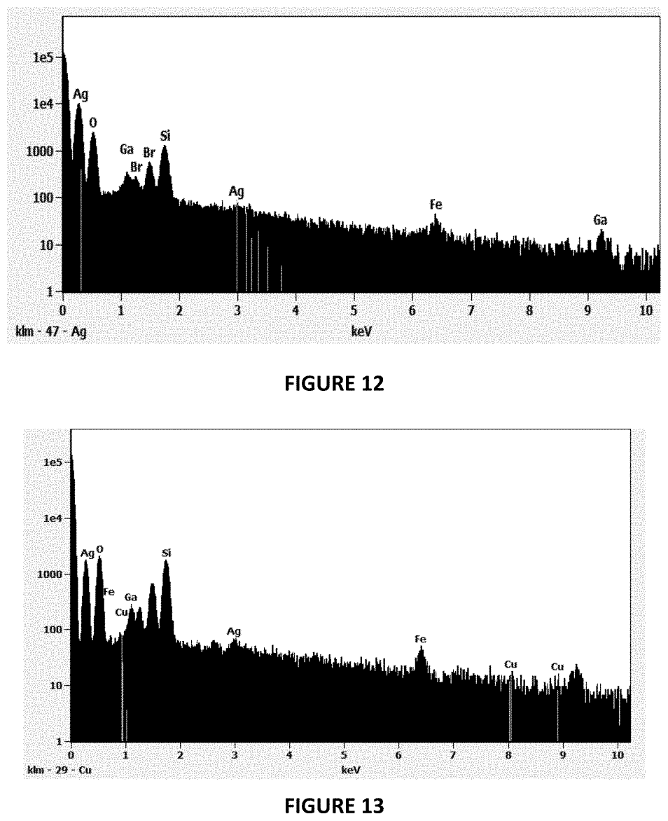

The molecular structure of sodium bentonite is Al₂H₂Na₂O₁₃Si₄. When this material is treated with AgNO₃ and Ga(NO₃)₃, it is expected that either Ag or Ga or both could replace the Na ions in the bentonite. The EDX spectrum of material 18 (FIG. 12) shows the presence of Si, O (both from bentonite), Ag and Ga, indicating that both Ag and Ga incorporate onto the bentonite. The weight percentage calculation indicates that the quantity of Ga is more than 2.6 times higher than Ag in the sample.

Nanolcay based antimicrobial additives which were synthesized using “dual metal combination” (materials 17-19) were subjected to antimicrobial efficacy testing (Table 8). Dual cation-bentonite combination systems involving BN-Ag-Cu (material 17), BN-Ag—Ga (material 18) and BN-Cu-Ga (material 19) did not show any antibacterial activity (MIC 128-1024 μg/mL).

TABLE 8
Antimicrobial activities in MIC (μg/mL) of dual metal nanoclay
systems against a series of clinically relevant bacteria

| | BN-Ag—Cu | BN-Ag—Ga | BN-Cu—Ga |
|---|---|---|---|
| Bacterial strain | (material 17) | (material 18) | (material 19) |
| MRSA(SA001) | 128 | 256 | >1024 |
| K. pneumoniae (KP001) | 128 | 256 | 1024 |
| A. baumannii (AB30) | 128 | 8 | >1024 |
| P. aeruginosa (PA07) | 128 | 256 | 1024 |
| E. cloacae (EB5) | 512 | 512 | >1024 |
| E. coli (E524) | 512 | 512 | >1024 |

### Example 8—Triple Combinations

### Triple Metals

In this experiment, we have incorporated three different metals, Ag, Cu and Ga using AgNO₃, CuSO₄ and Ga(NO₃)₃ salts onto the bentonite. The CEC ratio used was 1:0.66:0.66:0.66 (Bentonite, Ag, Cu and Ga salts respectively).

The EDX spectrum of BN-Ag-Cu-Ga sample (material 36) is shown in FIG. 13. It can be clearly seen that all three metals (Ag, Cu and Ga) are present in the sample. The weight ratio of the three metals in the final compound is 20:17:63 of Ag, Cu and Ga (excluding bentonite and other elements).

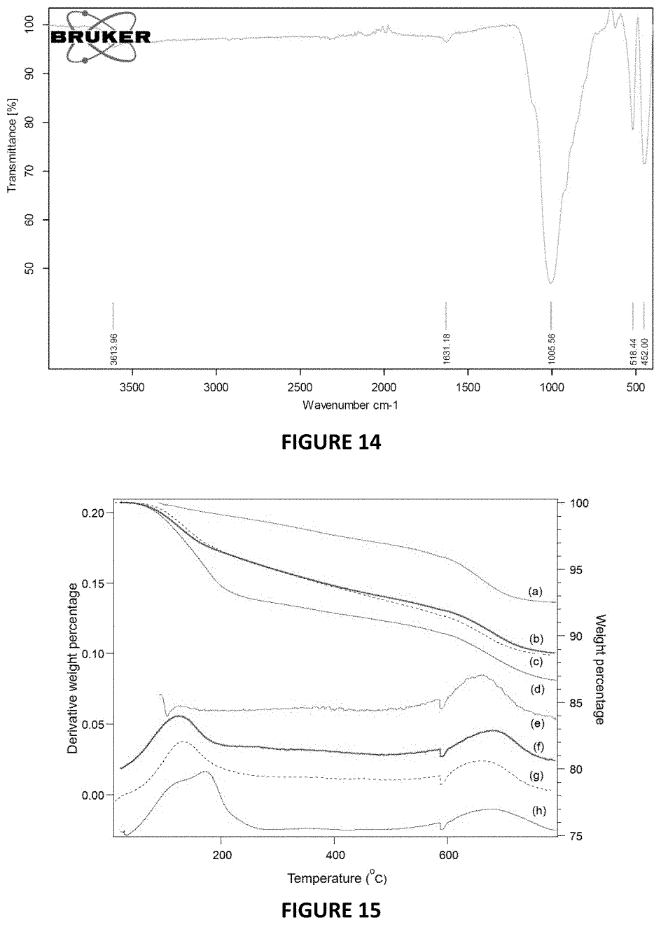

The FTIR spectrum of the material 36 looks similar to that of pure bentonite (FIG. 14). This is expected because the incorporated metals such as Ag, Cu and Ga do not show any stretching and bending vibration peaks.

FIG. 15 shows the TGA thermograms of bentonite incorporated individually with Ag, Cu and Ga and with all of them together. Both the weight loss and derivative weight loss thermograms of the BN-Ag-Cu-Ga sample look similar to that of BN-Ga. So, it is possible that the quantity of Ga present in the sample is much more compared to Ag and Cu. The weight ratio quantification through EDX also indicates that the quantity of Ga is at least three times higher than that of Ag and Cu.

Biological data: Nanolcay based antimicrobial additives which were synthesized using and triple metal combination (Ag—Cu—Ga, material 36) was subjected to antimicrobial efficacy testing (Table 9). The antimicrobial additive consisting of “triple metal ion combination” in bentonite (material 36) showed better antimicrobial efficacy against drug-resistant bacteria than the individual metal incorporated bentonites, except for BN-Ag.

TABLE 9
Antimicrobial activities in MIC (μg/mL) of dual metal and triple
metal nanoclay systems against a series of clinically relevant bacteria

| | BN-Ag | BN-Cu | BN-Ga | BN-Ag—Cu—Ga |
|---|---|---|---|---|
| Bacterial strain | (material 1) | (material 3) | (material 4) | (material 36) |
| MRSA(SA001) | 128 | 256 | >1024 | 256 |
| K. pneumoniae (KP001) | 256 | 1024 | >1024 | 256 |
| A. baumannii (AB30) | 256 | >1024 | 1024 | 256 |
| P. aeruginosa (PA07) | 256 | >1024 | >1024 | 256 |
| E. cloacae (EB5) | 128 | >1024 | >1024 | 1024 |
| E. coli (E524) | 256 | >1024 | >1024 | 512 |

### Zinc Pyrithione Based Triple Combinations

Zinc pyrithione was introduced into bentonite via mono (material 5), dual (materials 20 and 21) and triple (materials 37 and 38) cation exchange process with metal ions and quaternary ammonium compounds. Among them, bentonite combined zinc pyrithione (BN-ZnPY, material 5) showed good activity against some specific strains, viz., MRSA (SA001), K. pneumoniae (KP001), E. cloacae (EB5) and E. coli (E524). It was ineffective against Gram-negative P. aeruginosa and A. baumannii. Nanomaterials containing zinc pyrithione with copper (BN-Cu-ZnPY, material 20) and silver (BN-Ag-ZnPY, material 21) combination displayed good antibacterial activities against MRSA and K. pneumoniae. Whereas, zinc pyrithione in conjunction with silver/QAC (BN-Ag-BAC-ZnPY, material 37) and BN-Ag-BDMHAC-ZnPY, material 38) in a triple combination nanoclay system, showed good to moderate antimicrobial activities against a series of pathogenic bacteria (Table 10).

TABLE 10
Antimicrobial activities in MIC (μg/mL) of mono, dual and triple
nanoclay systems incorporated with metal-zinc pyrithione combinations

| | | BN-Cu- | BN-Ag- | BN-Ag- | BN-Ag- |
|---|---|---|---|---|---|
| | BN-ZnPY | ZnPY | ZnPY | BAC-ZnPY | BDMHAC-ZnPY |
| Bacterial strain | (material 5) | (material 20) | (material 21) | (material 37) | (material 38) |
| MRSA(SA001) | 4 | 2 | 4 | 8 | 8 |
| K. pneumoniae | 4 | 32 | 8 | 16 | 32 |
| (KP001) | | | | | |
| A. baumannii | 1024 | 128 | 32 | 32 | 64 |
| (AB30) | | | | | |
| P. aeruginosa | 1024 | 1024 | >1024 | 256 | 64 |
| (PA07) | | | | | |
| E. cloacae (EB5) | 8 | 64 | 16 | 32 | 64 |
| E. coli (E524) | 8 | 32 | 16 | 16 | 32 |

### Example 9—Dual Combinations with Different Nanoclays

In this set of experiments, a combination of chlorohexidine and neomycin has been incorporated onto different nanoclays including montmorillonite (MMT), halloysite and surface modified clay (SMC, Sigma Aldrich, Nanomer Clay, contains 0.5-5 wt. % aminopropyltriethoxysilane, 15-35 wt %).

### Montmorillonite+Chlorohexidine+Neomycin (MMT-CHL-NM)

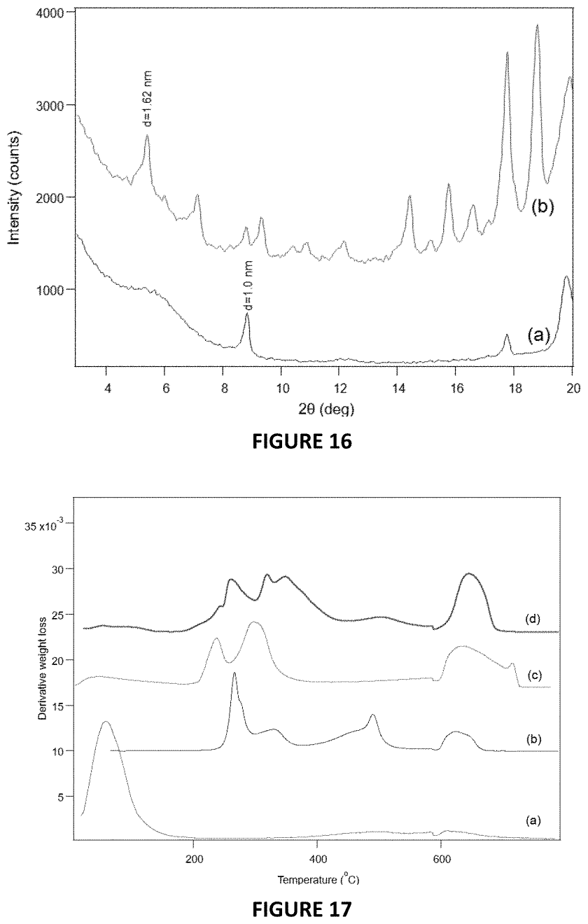

The XRD plots of montmorillonite (MMT) and MMT incorporated with chlorohexidine and neomycin (material 31) are shown in FIG. 16. As expected, the d-spacing of MMT has increased from 1.0 nm to 1.62 nm once the antimicrobials are incorporated. The peak at 1.0 nm corresponds to the basal plane spacing of unmodified montmorillonite clay that was shifted to lower 20 value after modification with the antimicrobials, indicating the intercalation of chlorohexidine and neomycin into the clay gallery (Mishra, A. K., et al. Characterization of surface-modified montmorillonite nanocomposites. Ceram. Int. 38, 929-934 (2012)).

Derivative weight loss thermogram curves for unmodified MMT, pure antimicrobials and material 31 are shown in the FIG. 17. The MMT shows a two-step decomposition profile with a very high char residue (87%). The individual antimicrobials such as chlorohexidine and neomycin sulfate and the sample show multi-step decomposition. The decomposition step below 100° C. for all the samples is attributed to desorption of water molecules from the external surface as well as inside the clay gallery, while other steps between 200° C. and 600° C. correspond to the decomposition of antimicrobial molecules ( ). The char residue of material 31 is 26%, indicating that the sample contains more than 60% antimicrobials. Also, the decomposition pattern of the material 31 looks like a combination of both that of neomycin sulfate and chlorohexidine, which shows that it may contain both antimicrobials.

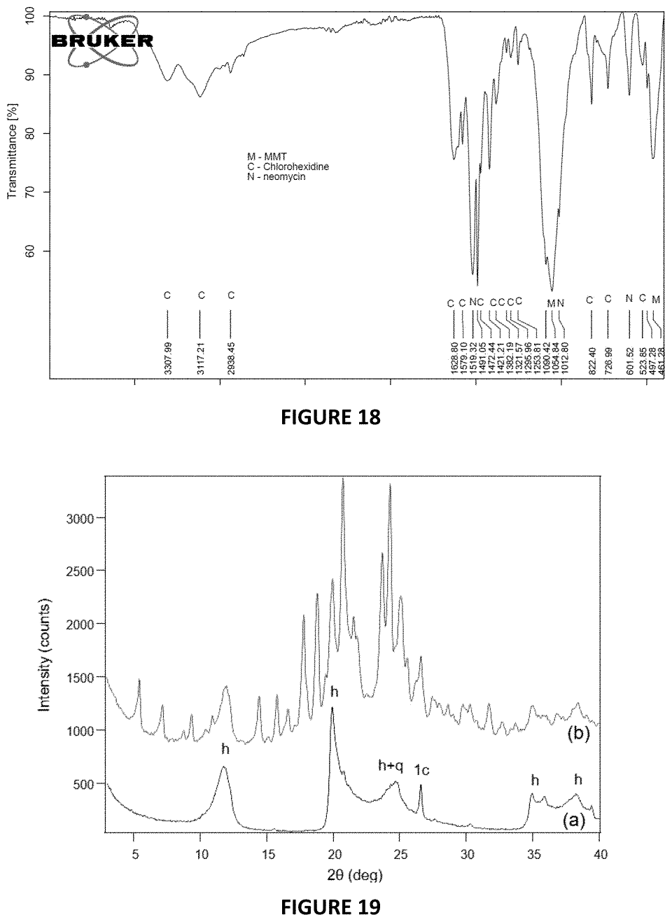

FIG. 18 shows the FTIR spectrum of MMT+NM-CHL (material 31) sample. The 460 cm′ deformation peak refers to Al—OH of the montmorillonite and the one at 1,051 cm⁻¹ is related to Si—O stretches (Tireli, A., et al. Fenton-like processes and adsorption using iron oxide-pillared clay with magnetic properties for organic compound mitigation. Environmental science and pollution research international 22, (2014)). The presence of all the above-mentioned peaks in the spectrum of material 31 indicates that the sample contains MMT, chlorohexidine and neomycin.

### Halloyasite+Chlorohexidine+Neomycin (HS-NM-CHL, Material 32)

The XRD spectrum of non-intercalated halloysite (FIG. 19) shows a basal reflection at 0.75 nm, which matches literature (Mellouk, S. et al. Applied Clay Science Intercalation of halloysite from Djebel Debagh (Algeria) and adsorption of copper ions. Appl. Clay Sci. 44, 230-236 (2009)). Once the antimicrobials are incorporated, the same has increased to 1.62 nm (for material 32).

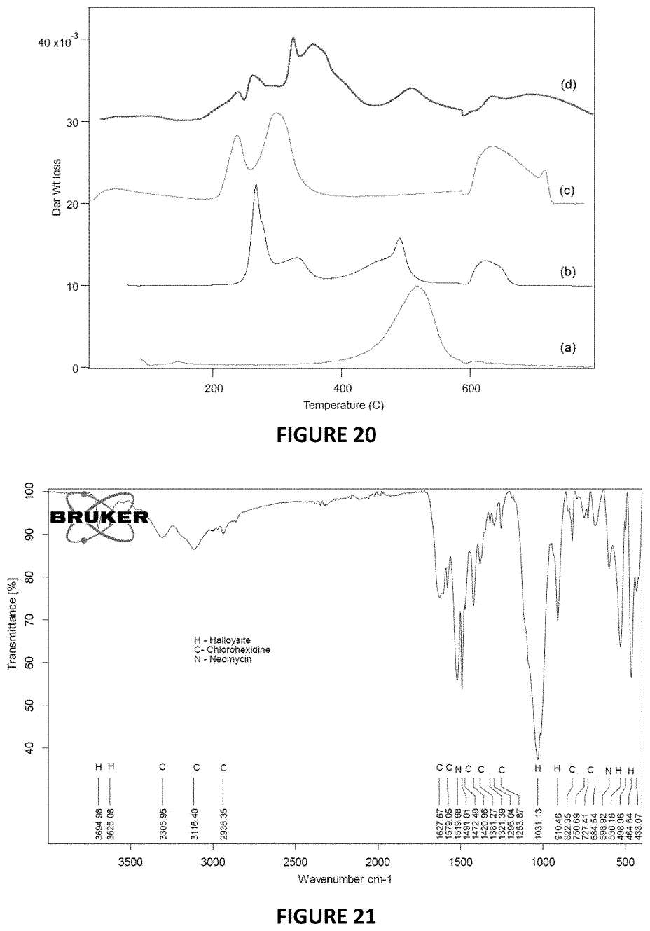

The TGA thermogram of pure halloysite shows one step degradation between 400 to 600° C. (FIG. 20a). This mass loss was assigned to the dehydroxylation of structural AlOH groups of halloysite (Chen, Y., et al. Preparation and antibacterial property of polyethersulfone ultrafiltration hybrid membrane containing halloysite nanotubes loaded with copper ions. Chem. Eng. J. 210, 298-308 (2012)). A slight peak before 100° C. is due to the desorption of water molecules from the halloysite. For the antimicrobial incorporated halloysite, char residue has decreased from 85% to 27%, indicating less thermally stable antimicrobials have been incorporated into the sample. Material 32 shows multi step decomposition, which includes all the individual decomposition patterns of halloysite, chlorohexidine and neomycin (FIG. 20b-d). Hence, the material 32 may contain all halloysite, chlorohexidine and neomycin in it.

FTIR spectrum of the halloysite asserts the following notes: the band at 3695 cm⁻¹ represents to the stretching vibration of the inner surface OH groups, while the band at 3625 cm⁻¹ represents to the stretching band of the inner groups. The inner surface OH groups are connected to the Al-centered octahedral sheets and form hydrogen bonds with the oxygen sheet in the next double layer (Gaaz, T. S., et al. A. The Impact of Halloysite on the Thermo-Mechanical Properties of Polymer Composites. Molecules 22, 1-20 (2017). The absorption peak at 1031 cm⁻¹ was the superposition of the stretching POC vibration at 1041 cm⁻¹ and the absorption peak at 1030 cm⁻¹ of raw HNTs (Wang, Z., et al. Preparation and antifouling property of polyethersulfone ultrafiltration hybrid membrane containing halloysite nanotubes grafted with MPC via RATRP method. Desalination 344, 313-320 (2014)). Apart from the halloysite bands, the spectrum also contains the peaks from both chlorohexidine and neomycin indicating their presence in the sample (FIG. 21).

### Surface Modified Clay+Chlorohexidine+Neomycin (SMC-NM-CHL, Material 30)

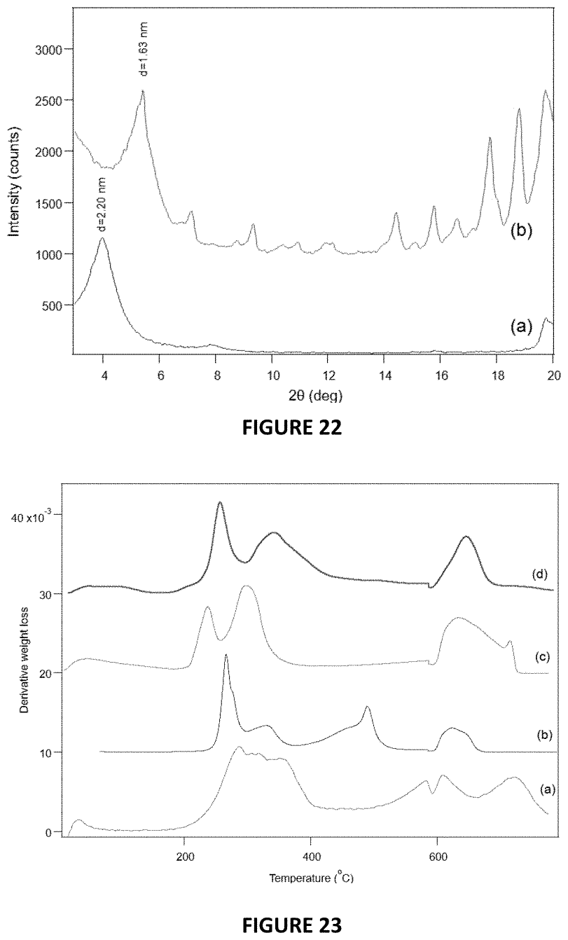

The interlayer d-spacing for the surface modified montmorillonite is 2.20 nm since it had been already intercalated with long chain octadecylamine (>35%). However, the d-spacing decreases to 1.63 nm after treating this clay with chlorohexidine and neomycin (FIG. 22).

The thermogram of SMC show multi-step decomposition with a major decomposition occurs between 200-400° C. (FIG. 23a). This is due to the degradation of the surface modifier, octadecylamine present in the sample. The char residue of SMC is 69%, which is lower than the unmodified MMT (87%). This excess weight loss of the SMC could be attributed to the loss of surface modifiers. Material 30 shows degradation peaks of both chlorohexidine and neomycin as expected (FIG. 23b-d). Also, sample 30 does not show the degradation peaks of pure SMC, which highlights the fact that the surface modifier (octadecylamine) has been removed from the sample. And the char residue of sample 30 is 34%, indicating the higher presence of the antimicrobials in it.

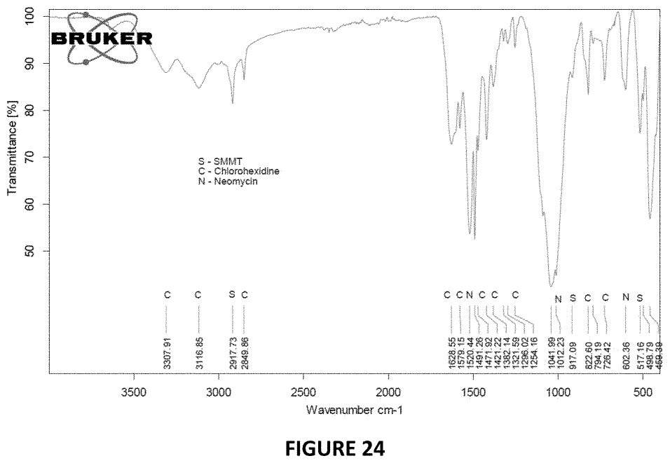

The FTIR spectrum of SMC-NM-CHL (material 30) sample is shown in FIG. 24. The large band at 1042 cm⁻¹ and small band at 917 cm⁻¹ are assigned to Si—O stretching and Al—Al—OH (present on the edges of the clay platelets) hydroxyl bending vibrations of MMT. The peaks at 525 and 456 cm⁻¹ are associated with Si—O—Al and Si—O—Si bending vibrations of SMC, respectively (Mishra, A. K., et al. Characterization of surface-modified montmorillonite nanocomposites. Ceram. Int. 38, 929-934 (2012)). In addition to these SMC peaks, the sample also contains the bands from both chlorohexidine and neomycin, which proves their presence in the sample.

Modification of d-Spacing

Table 11 shows the d-spacing of various nanoclays (bentonite, halloysite, montmorillonite and surface modified nanoclay) that were modified with various antimicrobials through mono, dual and triple cationic exchange processes in accordance with the present invention. As can be seen, the d-spacing changes depending on the type and size of the exchangeable antimicrobial cations.

TABLE 11
d-spacing of the cation exchanged nanoclays taken by XRD

| Material (Material number) | d-spacing |
|---|---|
| BN | 1.30 |
| HS | 0.75 |
| MMT | 1.00 |
| S-MMT | 2.20 |
| BN-Ag (1) | 1.41 |
| BN-Cu (3) | 1.25 |
| BN-Ga (4) | 1.44 |
| BN-BAC (6) | 2.10 |
| BN-Ag-BAC (22) | >2.94 |
| BN-BDMHAC (7) | 1.78 |
| BN-Ag-BDMHAC (24) | 1.83 |
| BN-BDMTAC (8) | 1.72 |
| BN-Ag-BDMTAC (25) | 1.70 |
| BN-Cu-BDMHAC (26) | 1.80 |
| BN-NM (13) | 1.43 |
| BN-CHL (12) | 1.49 |
| BN-NM-CHL (29) | 1.62 |
| SMC-NM-CHL (30) | 1.63 |
| MMT-NM-CHL (31) | 1.62 |
| HS-NM-CHL (32) | 1.62 |
| BN-EDTA (16) | 1.42 |
| BN-Ag-EDTA (34) | 1.43 |
| BN-BAC-EDTA (35) | 2.94 |
| BN-ZnPy (5) | 1.42 |
| BN-Ag—Cu (17) | 1.28 |
| BN-Ag—Ga (18) | 1.43 |
| BN-Cu—Ga (19) | 1.40 |
| BN-Ag—Cu—Ga (36) | 1.40 |

### Biological Data

Impact of nanoclay architecture on antimicrobial activities was also explored by evaluating antimicrobial efficacies of Surface modified clay-Neomycin-Chlorohexidine (SMC-NM-CHL, material 30), Montmorillonite-Neomycin-Chlorohexidine (MMT-NM-CHL, material 31) and Halloysite-Neomycin-Chlorohexidine (HS-NM-CHL, material 32). All four different kinds of nanoclay materials embedded with the same antimicrobial combination, showed remarkable synergy antimicrobial efficacies against drug-resistant bacteria (Table 12). Whereas single antimicrobial containing bentonite materials, Bentonite-Chlorohexidine (BN-CHL, material 12) and Bentonite-Neomycin (BN-NM, material 13) showed primarily no or moderate activity against all the strains tested (Table 12). For example, individually incorporated antimicrobials into bentonite, materials 12 and 13 displayed MIC 64 and >1024 μg/mL respectively against clinically relevant P. aeruginosa (PA07). Whereas, combined integration of neomycin and chlorhexidine into bentonite system (material 29) resulted in MIC of 2 μg/mL (524 to 64 folds higher antimicrobial efficacy than individual components) against P. aeruginosa (PA07).

TABLE 12
Antimicrobial activities in MIC (μg/mL) of NM, CHL, SMC-
NM-CHL (material 30), MMT-NM-CHL (material 31) and HS-NM-CHL
(material 32) against a series of clinically relevant bacteria

| | | | SMC- | MMT- | HS-NM- |
|---|---|---|---|---|---|
| | Neomycin | Chlorhexidine | NM-CHL | NM-CHL | CHL |
| | (NM) | (CHL) | (material 30) | (material 31) | (material 32) |
| P. aeruginosa (PA07) | 128 | 16 | 2 | 2 | 2 |
| K. pneumoniaec(KP001) | 2 | <0.5 | 0.5 | 0.5 | 0.5 |
| A. baumannii (AB30) | 8 | 256 | 16 | 16 | 16 |
| E. coli (E524) | 2 | 2 | 2 | 2 | 1 |
| E. cloacae (EB5) | 4 | 8 | 2 | 2 | 2 |
| MRSA (SA004) | 4 | 8 | 2 | 2 | 2 |

Antimicrobial Surface Studies: The study of surface bactericidal activity of BN-Ag-BAC (material 22) against a range of bacterial isolates displayed superior R value (log reduction) and corresponding % reduction (Table 13). To determine the antimicrobial activity of material 22 coated surfaces, JIS Z 2801: 2010 standard protocol was followed. The values obtained demonstrated excellent antimicrobial activity of the coated surfaces compared to control coated (Lab control, LDPE) surfaces. A well-accepted indicator of antibacterial activity is the R value, which is the Log₁₀ (B-C), where B is the CFUs on control coated surfaces, and C is the CFUs on test surfaces (material 22). The antimicrobial efficacy of material 22 treated surfaces demonstrated as CFUs present, antimicrobial activity (R) and microbial kill (% reduction). The tested pathogen list contains major human pathogenic bacteria, including aerobic and facultative anaerobic, gram +ve and gram −ve strains. Many of these strains included here are common opportunistic pathogens and may demonstrate multi-drug resistance. The antimicrobial efficacy of material 22 has been tested against Listeria monocytogenes, bacterial strain involved in food contamination resulting in serious human infection. It is worth to note that material 22 treated surfaces also demonstrated superior antimicrobial activity against two common human mycopathogenic strains, Aspergillus flavus and Candida albicans. These fungi are opportunistic human pathogens, and the former, is known to contaminate cereal grains and legumes, and producing mycotoxins, that are toxic to mammals when consumed.

TABLE 13
Antibacteria effectiveness of material 22 against various bacteria

| | | Log₁₀ | | | R = Log₁₀ | |
|---|---|---|---|---|---|---|
| | CFUs | Control | CFUs | Log₁₀ | (B) − | Microbial |
| Microbial strains | Control | surface | (46) | Of (46) | Log₁₀ | kill (% |
| ATCC # | surface | (B) | surface | (C) | (C) | reduction) |
| Klebsiella | 1.51 × 10⁸ | 8.17 | 1740 | 3.24 | 4.93 | 99.998 |
| pneumoniae | | | | | | |
| ATCC 4352 | | | | | | |
| Acinetobacter | 9.80 × 10⁷ | 7.99 | 8100 | 3.90 | 4.09 | 99.991 |
| baumannii | | | | | | |
| ATCC 19606 | | | | | | |
| Staphylococcus | 9.90 × 10⁸ | 8.99 | <10 | <1 | >7.99 | >99.99999 |
| aureus | | | | | | |
| ATCC 6538 | | | | | | |
| Escherichia coli | 8.10 × 10⁸ | 8.9 | 30 | 1.47 | 7.43 | 99.99999 |
| ATCC 25922 | | | | | | |
| Pseudomonas | 8.80 × 10⁸ | 8.94 | 430 | 2.63 | 6.31 | 99.9999 |
| aeruginosa | | | | | | |
| ATCC 9027 | | | | | | |
| Enterobacter | 9.20 × 10⁸ | 8.96 | 430 | 2.63 | 6.33 | 99.9999 |
| aerogenes | | | | | | |
| ATCC 13048 | | | | | | |
| Enterococcus faecium | 1.15 × 10⁸ | 8.06 | 160 | 2.20 | 5.86 | 99.999 |
| ATCC 8459 | | | | | | |
| Shigella flexneri | 8.80 × 10⁸ | 8.94 | 240 | 2.38 | 6.56 | 99.9999 |
| ATCC 9199 | | | | | | |
| Clostridium | 1.62 × 10⁸ | 8.20 | 400 | 2.60 | 5.60 | 99.999 |
| perfringens | | | | | | |
| ATCC 7939 | | | | | | |
| Corynebacterium | 7.50 × 10⁷ | 7.87 | <10 | <1 | >6.87 | >99.9999 |
| minutissimum | | | | | | |
| ATCC 23348 | | | | | | |
| Listeria | 1.75 × 10⁸ | 8.24 | <10 | <1 | >7.24 | >99.99999 |
| monocytogenes | | | | | | |
| ATCC 23074 | | | | | | |
| Salmonella typhi | 1.69 × 10⁸ | 8.22 | <10 | <1 | >7.22 | >99.99999 |
| ATCC 10749 | | | | | | |
| Serratia marcescens | 1.69 × 10⁸ | 8.22 | 800 | 2.90 | 5.32 | 99.999 |
| ATCC 14756 | | | | | | |
| Aspergillus flavus | 1.40 × 10⁸ | 8.14 | <10 | <1 | >7.14 | >99.99999 |
| ATCC 9643 | | | | | | |
| Candida albicans | 6.90 × 10⁸ | 8.14 | <10 | <1 | >7.83 | >99.99999 |
| ATCC 10231 | | | | | | |

Altogether these examples support the utility of the present invention and support the advantages associated with the antimicrobial nanoclays defined herein, including facile synthesis, long-term stability, water insolubility, nontoxicity and broad-spectrum biocidal activity over short contact times.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “an antimicrobial” includes one or more of such antimicrobials and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

What is claimed is:

1. An antimicrobial nanoclay comprising:
a phyllosilicate clay; and
two or more cationic antimicrobials, wherein said two or more cationic antimicrobials are individually selected from the group consisting of a quaternary ammonium compound, a metal ion-containing compound, a cationic chelating agent, a cationic amino acid-based surfactant, a cationic antibiotic, and a cationic compound having a guanidine moiety.

2. The antimicrobial nanoclay according to claim 1 wherein the phyllosilicate clay is selected from the group consisting of: clays of montmorillonite, kaolinite, bentonite, smectite, hectorite, sepiolite, gibbsite, dickite, nacrite, saponite, halloysite, vermiculite, mica type, surface modified clay, chlorite, illite kalonite-serpentine, nontronite, attapulgite and mixtures thereof.

3. The antimicrobial nanoclay according to claim 1, wherein the nanoclay is selected from the group consisting of bentonite, montmorillonite, kalonite-serpentine, and halloysite.

4. (canceled)

5. The antimicrobial nanoclay according to claim 1, wherein the metal ion-containing compound is selected from the group of compounds comprising a metal ion consisting of silver (Ag), titanium (Ti), cobalt (Co), nickel (Ni), zinc (Zn), molybdenum (Mo), gallium (Ga), copper (Cu), zirconium (Zr), tin (Sn), lead (Pb), and iron (Fe).

6. The antimicrobial nanoclay according to claim 1, wherein the quaternary ammonium compound is selected from the group consisting of a benzalkonium compound having alkyl chains C8 to C18; a quaternary ammonium compound having an aromatic ring with hydrogen and chlorine, methyl and ethyl groups; a quaternary ammonium compound having a dialkylmethyl amino with twin chains; and a polymeric quaternary ammonium compound.

7. The antimicrobial nanoclay according to claim 1, wherein the cationic chelating agent is selected from the group consisting of ethyleneglycoltetraacetate (EGTA), ethylenediaminetetraacetate (EDTA), deferrioxamine B (DFO, desferal), D-penicillamine (DPA), 1,10-phenanthroline (phen), and zinc pyrithione.

8. The antimicrobial nanoclay according to claim 1, wherein the antibiotic is selected from the classes consisting of an aminoglycoside, an ansamycin, a carbacephem, a carbapenem, a cephalosporin, a glycopeptide, a lincosamide, a lipopeptide, a macrolide, a monobactams, a nitrofuran, an oxazolidinone, a penicillin, a polypeptide, a quinoline, a fluoroquinoline, a sulfonamide, and a tetracycline.

9. (canceled)

10. The antimicrobial nanoclay according to claim 1, wherein the guanidine functionality containing group is chlorhexidine gluconate (CHG), polyhexamethylene guanidine hydrochloride, guanidine hydrochloride, proguanil, cycloguanil, chlorproguanil, metformin, famotidine, nemaucing, rhodostreptomycin A, rhodostreptomycin B, synoxaazolidinone A, synoxaazolidinone B or a chemical derivative thereof.

11. The antimicrobial nanoclay according to, wherein the antimicrobial nanoclay comprises two or more cationic antimicrobials from at least two different groups or classes of compounds.

12. The antimicrobial nanoclay according to claim 11 wherein said at least two different cationic antimicrobials are independently selected from the group consisting of silver, copper, gallium, zinc pyritione, benzalkonium chloride, polymyxin E, niclosamide, ethylenediaminetetraacetic acid, benzyldimethyltetradecylammonium chloride, neomycin, and chlorohexidine.

13. The antimicrobial nanoclay according to claim 1, wherein the antimicrobial nanoclay comprises at least three different cationic antimicrobials.

14. The antimicrobial nanoclay according to claim 13, wherein said at least three different cationic antimicrobials are from at least three different groups or classes of compounds.

15. (canceled)

16. (canceled)

17. The antimicrobial nanoclay according to claim 1, wherein the antimicrobial nanoclay is any one from Material Number 17 to 144:

| Material Number | R | R₁ | R₂ | R₃ |
|---|---|---|---|---|
| 17 | BN | Ag | Cu | — |
| 18 | BN | Ag | Ga | — |
| 19 | BN | Cu | Ga | — |
| 20 | BN | Cu | ZnPy | — |
| 21 | BN | Ag | ZnPy | — |
| 22 | BN | Ag | BAC | — |
| 23 | BN | Cu | BAC | — |
| 24 | BN | Ag | BDMHAC | — |
| 25 | BN | Ag | BDMTAC | — |
| 26 | BN | Cu | BDMHAC | — |
| 27 | BN | Cu | BDMTAC | — |
| 28 | BN | BAC | BDMHAC | — |
| 29 | BN | NM | CHL | — |
| 30 | SMC | NM | CHL | — |
| 31 | MMT | NM | CHL | — |
| 32 | HS | NM | CHL | — |
| 33 | BN | PMX | NCL | — |
| 34 | BN | Ag | EDTA | — |
| 35 | BN | BAC | EDTA | — |
| 36 | BN | Ag | Cu | Ga |
| 37 | BN | Ag | BAC | ZnPy |
| 38 | BN | Ag | BDMHAC | ZnPy |
| 39 | BN | BAC | BDMHAC | BDMTAC |
| 40 | BN | NM | CHL | EDTA |
| 46 | MMT | Ag | Cu | — |
| 47 | MMT | Ag | Ga | — |
| 48 | MMT | Cu | Ga | — |
| 49 | MMT | Cu | ZnPy | — |
| 50 | MMT | Ag | ZnPy | — |
| 51 | MMT | Ag | BAC | — |
| 52 | MMT | Cu | BAC | — |
| 53 | MMT | Ag | BDMHAC | — |
| 54 | MMT | Ag | BDMTAC | — |
| 55 | MMT | Cu | BDMHAC | — |
| 56 | MMT | Cu | BDMTAC | — |
| 57 | MMT | BAC | BDMHAC | — |
| 58 | MMT | Ag | EDTA | — |
| 59 | MMT | BAC | EDTA | — |
| 60 | MMT | PMX | NCL | — |
| 61 | MMT | Ag | Cu | Ga |
| 62 | MMT | Ag | BAC | ZnPy |
| 63 | MMT | Ag | BDMHAC | ZnPy |
| 64 | MMT | BAC | BDMHAC | BDMTAC |
| 65 | MMT | NM | CHL | EDTA |
| 66 | SMC | Ag | Cu | — |
| 67 | SMC | Ag | Ga | — |
| 68 | SMC | Cu | Ga | — |
| 69 | SMC | Cu | ZnPy | — |
| 70 | SMC | Ag | ZnPy | — |
| 71 | SMC | Ag | BAC | — |
| 72 | SMC | Cu | BAC | — |
| 73 | SMC | Ag | BDMHAC | — |
| 74 | SMC | Ag | BDMTAC | — |
| 74 | SMC | Cu | BDMHAC | — |
| 75 | SMC | Cu | BDMTAC | — |
| 76 | SMC | BAC | BDMHAC | — |
| 77 | SMC | Ag | EDTA | — |
| 78 | SMC | BAC | EDTA | — |
| 79 | SMC | PMX | NCL | — |
| 80 | SMC | Ag | Cu | Ga |
| 81 | SMC | Ag | BAC | ZnPy |
| 82 | SMC | Ag | BDMHAC | ZnPy |
| 83 | SMC | BAC | BDMHAC | BDMTAC |
| 84 | SMC | NM | CHL | EDTA |
| 85 | HS | Ag | Cu | — |
| 86 | HS | Ag | Ga | — |
| 87 | HS | Cu | Ga | — |
| 88 | HS | Cu | ZnPy | — |
| 89 | HS | Ag | ZnPy | — |
| 90 | HS | Ag | BAC | — |
| 91 | HS | Cu | BAC | — |
| 92 | HS | Ag | BDMHAC | — |
| 93 | HS | Ag | BDMTAC | — |
| 93 | HS | Cu | BDMHAC | — |
| 94 | HS | Cu | BDMTAC | — |
| 95 | HS | BAC | BDMHAC | — |
| 96 | HS | Ag | EDTA | — |
| 97 | HS | BAC | EDTA | — |
| 98 | HS | PMX | NCL | — |
| 99 | HS | Ag | Cu | Ga |
| 100 | HS | Ag | BAC | ZnPy |
| 101 | HS | Ag | BDMHAC | ZnPy |
| 102 | HS | BAC | BDMHAC | BDMTAC |
| 103 | HS | NM | CHL | EDTA |
| 104 | KL | Ag | Cu | — |
| 105 | KL | Ag | Ga | — |
| 106 | KL | Cu | Ga | — |
| 107 | KL | Cu | ZnPy | — |
| 108 | KL | Ag | ZnPy | — |
| 109 | KL | Ag | BAC | — |
| 110 | KL | Cu | BAC | — |
| 111 | KL | Ag | BDMHAC | — |
| 112 | KL | Ag | BDMTAC | — |
| 113 | KL | Cu | BDMHAC | — |
| 114 | KL | Cu | BDMTAC | — |
| 115 | KL | BAC | BDMHAC | — |
| 116 | KL | Ag | EDTA | — |
| 117 | KL | BAC | EDTA | — |
| 118 | KL | PMX | NCL | — |
| 119 | KL | NM | CHL | — |
| 120 | KL | Ag | Cu | Ga |
| 121 | KL | Ag | BAC | ZnPy |
| 122 | KL | Ag | BDMHAC | ZnPy |
| 123 | KL | BAC | BDMHAC | BDMTAC |
| 124 | KL | NM | CHL | EDTA |
| 125 | LP | Ag | Cu | — |
| 126 | LP | Ag | Ga | — |
| 127 | LP | Cu | Ga | — |
| 128 | LP | Cu | ZnPy | — |
| 129 | LP | Ag | ZnPy | — |
| 130 | LP | Ag | BAC | — |
| 131 | LP | Cu | BAC | — |
| 132 | LP | Ag | BDMHAC | — |
| 133 | LP | Ag | BDMTAC | — |
| 134 | LP | Cu | BDMHAC | — |
| 135 | LP | Cu | BDMTAC | — |
| 136 | LP | BAC | BDMHAC | — |
| 137 | LP | Ag | EDTA | — |
| 138 | LP | BAC | EDTA | — |
| 139 | LP | PMX | NCL | — |
| 140 | LP | NM | CHL | — |
| 141 | LP | Ag | BAC | ZnPy |
| 142 | LP | Ag | BDMHAC | ZnPy |
| 143 | LP | BAC | BDMHAC | BDMTAC |
| 144 | LP | NM | CHL | EDTA |

wherein R represents the phyllosilicate nanoclay, R₁ represents a first antimicrobial, R₂ represents a second antimicrobial, R₃ represents a third antimicrobial; and
wherein BN=Bentonite; SMC=Surface modified clay; MMT=Montmorillonite; HS=Halloysite; KL=Kaolinite; LP=Laponite; Ag=Silver; Cu=Copper; Ga=Gallium; ZnPy=Zinc pyrithione; BAC=Benzalkonium chloride; BDMHAC=Benzyldimethylhexadecylammonium chloride; BDMTAC=Benzyldimethyltetradecylammonium chloride; NM=Neomycin; CHL=Chlorohexidine; PMX=Polymyxin E; NCL=Niclosamide; EDTA=Ethylenediaminetetraacetic acid.

18. The antimicrobial nanoclay according to claim 1, wherein the first antimicrobial agent is silver and the second antimicrobial agent is Benzalkonium chloride (BAC).

19. The antimicrobial nanoclay according to claim 1, wherein the first antimicrobial agent is silver and the second antimicrobial agent is Benzyldimethylhexadecylammonium chloride (BDMHAC).

20. The antimicrobial nanoclay according to claim 1, wherein the first antimicrobial agent is silver and the second antimicrobial agent is Benzyldimethyltetradecylammonium chloride (BDMTAC).

21. The antimicrobial nanoclay according to claim 1, wherein the first antimicrobial agent is Benzalkonium chloride (BAC) and the second antimicrobial agent is Ethylenediaminetetraacetic acid (EDTA).

22. The antimicrobial nanoclay according to claim 1, wherein the first antimicrobial agent is Chlorhexidine and the second antimicrobial agent is Neomycin, or wherein the first antimicrobial agent is Polymyxin and the second antimicrobial agent is Niclosamide.

23. (canceled)

24. The antimicrobial nanoclay according to claim 1, further comprising at least one additive for enhancing at least one of the following properties of the antimicrobial nanoclay: mechanical properties, thermal properties, electrical properties, barrier properties, rheology properties, scavenging properties, exfoliation properties, fire retardant properties, plasticize properties, permeability properties and color properties.

25. An article of manufacture comprising an antimicrobial nanoclay as defined in claim 1.

26. (canceled)

27. A method for conferring antimicrobial activity to a surface, comprising contacting said surface with an antimicrobial nanoclay as defined in claim 1.

28.-31. (canceled)

32. A method for preparing an antimicrobial nanoclay as defined in claim 1, comprising the steps of:
providing an aqueous solvent having a pH of between about 3 to about 7;
dispersing a phyllosilicate clay in the aqueous solvent to obtain a dispersed clay solution; and
mixing two or more cationic antimicrobials into the clay solution to obtain an antimicrobial clay mixture;
wherein said antimicrobials are selected from the group consisting of a quaternary ammonium compound, a metal ion-containing compound, a cationic chelating agent, a cationic amino acid-based surfactant, a cationic antibiotic, and a cationic compound having a guanidine moiety

33.-40. (canceled)
